(19) United States
(12) Patent Application Publication
(10) Pub. No.: US 2018/0028088 A1
(43) Pub. Date: Feb. 1, 2018

(54) SYSTEMS AND METHODS FOR MEDICAL PROCEDURE MONITORING

(71) Applicants: UNIVERSITY OF HOUSTON SYSTEM, Houston, TX (US); THE METHODIST HOSPITAL, Houston, TX (US)

(72) Inventors: Marc GARBEY, Houston, TX (US); Ahmet OMURTAG, Houston, TX (US); Barbara Lee BASS, Houston, TX (US); Brian James DUNKIN, Houston, TX (US)

(73) Assignees: UNIVERSITY OF HOUSTON SYSTEM, Houston, TX (US); THE METHODIST HOSPITAL, Houston, TX (US)

(21) Appl. No.: 15/553,662

(22) PCT Filed: Feb. 26, 2016
(86) PCT No.: PCT/US16/19713

§ 371 Date: Aug. 25, 2017

Related U.S. Application Data
(60) Provisional application No. 62/126,181, filed on Feb. 27, 2015.

(51) Int. Cl. A61B 5/0476; A61B 90/00; A61B 5/16; A61B 5/00; A61B 5/04

(52) U.S. Cl. CPC A61B 5/0476; A61B 5/0006; A61B 5/04012; A61B 5/16; A61B 5/7405; A61B 90/37

(57) ABSTRACT

Systems and methods for monitoring medical procedures. Particular embodiment's relate to monitoring medical procedures performed in operating room environments through the use of various types of sensors, including for example, wireless electroencephalography (EEG) monitoring systems. Presented are systems and methods directed to a monitor medical procedures, including in particular the mental state of medical personnel associated with such procedures. Exemplary embodiment's of the present disclosure relate to systems and methods for monitoring medical procedures. Particular embodiment's relate to monitoring medical procedures performed in operating room environments through the use of various types of sensors, including for example, wireless electroencephalography (EEG) monitoring systems.

### CROSS-REFERENCE TO RELATED APPLICATIONS

This application claims priority to U.S. Provisional Patent Application Ser. No. 62/126,181 filed Feb. 27, 2015, the contents of which are incorporated herein by reference.

### TECHNICAL FIELD

Exemplary embodiments of the present disclosure relate to systems and methods for monitoring medical procedures. Particular embodiments relate to monitoring medical procedures performed in operating room environments through the use of various types of sensors, including for example, wireless electroencephalography (EEG) monitoring systems.

### BACKGROUND

Safety surveillance of procedure in the hospital Operating Rooms (OR) can benefit dramatically from the understanding of the cognitive dynamic of the surgery team coupled to a noninvasive tracking of the procedural steps.

For a surgery team, standard procedures (including for example, cholecystectomy) repeated multiple times during the day may generate excessive fatigue leading to surgical errors. In addition, complex lengthy procedures (including for example, organ transplants) may generate excessive stress and frustrations leading to surgical error as well.

Such errors may impact the surgical outcome for the patient in dramatic ways. Furthermore, such surgical errors are devastating for the surgical team.

A system is disclosed herein that maintains a cognitive awareness of the surgical team as well as tracks key events and maneuvers on the procedure at multiple levels. Exemplary embodiments may monitor cognitive awareness via a portable wireless EEG device worn by operating room individuals on the surgical team. Both channels of information (e.g. cognitive and procedural monitoring) can be combined to provide robust safety measures. Further interpretation of the EEG signal, especially with noninvasive sensors (such as wearable light and low cost dry sensor), carries a significant level of uncertainties, and might be rather sensitive to individual. It is advocated that coupling both channel of information end up into a robust method to acquire cognitive awareness of the hospital operation room and improve safety.

In addition, optimum management of multiple hospital operating rooms (OR) is a complex problem. For example, a large hospital such as the Houston Methodist Hospital has approximately seventy active ORs with a large number of procedures per day and per OR that need to be scheduled several weeks in advance. Each procedure requires gathering a team led by a surgeon for a specific block of time in the OR. But even the most standard procedure, such as a cholecystectomy (which account for approximately 600,000 cases per year in the United States), can exhibit a significant variation in time duration. It is often the case that multiple OR scheduling must be done under uncertainties on time duration. Some procedures may lead to fairly unpredictable events, such as unexpected bleeding or additional work that requires more time, and possibly more personnel and equipment.

While the OR is a complex, high technology setting, there is still not an automatic feedback loop between the surgical team and the OR system to allow real time adjustment of previously made decisions in scheduling. It is believed that effective OR awareness could provide early signs of problems that can allow the OR management to reallocate resources in a more efficient way. For example, a first step could be to have an OR that has tracking capability of all key events in order to assess the working flow in multiple ORs and build a statistical model that can be used to rationalize decision making and resource allocation. While there have been numerous works investigating this issue, it seems that there has been no practical solution implemented yet to provide the necessary data for this endeavor.

It has been recognized that OR time is one of the most significant budget expenses in a modern hospital. It is also recognized that delays in OR procedures due to lapses in scheduling and/or OR resources availability have been responsible for increased failures in surgery outcome.

Previous investigators (e.g. University of Iowa Prof. Franklin Dexter) have provided an extensive bibliography on OR management under various aspect such as rational on economics, algorithmic methods to optimize the management, and necessary tools to predict surgery procedure duration. However, such disclosures do not provide systems and methods as disclosed herein utilizing appropriate sensors, modeling, and computer processing implementation.

Previous investigations into OR management optimization typically reviewed OR allocation several days prior to surgery. The input flow of OR procedures to be achieved as well as the resources available (staff, OR, equipment, etc. . . . ) to do the work are assumed to be known. In previous investigations, the problem is typically formalized mathematically and solved with some optimization algorithm. In addition, several assumptions are often made on the level of complexity of the problem, depending on the time scale, number of ORs and/or types of surgery. It is assumed that the data available—such as expected time for surgery, patient and staff availability—can be either deterministic or probabilistic with a certain level of uncertainties. In typical previous investigation, the panel of mathematical methods to solve the problem encompasses linear integer programming, petri nets, stochastic optimization, etc. Validation is often based either on simulation tools or true comparison between different methods of scheduling in clinical conditions. However, this work is often based on tedious data acquisition that is done manually, which can be an obstacle to going further and deeper in the OR management field. Exemplary embodiments disclosed herein provide systems and methods to address such issues.

Previous investigations into predicting OR task durations typically rely on extensive collection of data acquisition on OR activities. In such cases, one needs to decide about the level of details used in the description of the procedure, which can result in a statistical model that might be valid for the specific category of intervention only. The reliability of such a statistical model depends on the normalization of the procedure and the quality of service at the hospital. This in turn depends on the standard of the surgical team and might be available only to large volume procedure that offers enough reproducibility.

Prior techniques that have been used to record and annotate the OR activities include a video camera mounted in the light that is above the OR table. In addition, sometimes a fixed video camera may also be mounted on the wall of the OR. For minimally invasive surgery, the video output of the endoscope camera may also be projected and/or recorded. There have been numerous works in computer vision then that either concentrate on following the motion and movements of the surgical team in the OR, or the laparoscopic instrument in the abdominal cavity.

It is also possible to analyze the motion of the hand of the surgeon during the procedure. There is continuous progress made on pattern recognition. It is however, quite difficult to get such methods working with sufficient and consistent accuracy. A primary reason is that there is typically significant variability with in people motion. Tracking a specific event or individual may become unfeasible, due to obstruction of view, or with staff moving in and out of multiple ORs. Accordingly, a computer vision method for OR function tracking is presented with significant obstacles. Exemplary embodiments disclosed herein include systems and method based on distributed sensors to track specific events to address these and other issues.

Previous investigations have also addressed the tracking of OR functions at the surgical tool level. The field of laparoscopic surgery of large volume minimally invasive surgery is one example. In addition, extensive study based on pattern recognition of tools in that view has also been published. Furthermore, RFID tracking of instruments has been a popular solution. However, the OR environment is not favorable to this technology. Similarly, using a bar code on each laparoscopic instrument is also not considered a robust solution.

Therefore, a need in the art exists for a minimally intrusive, yet robust, systems and methods to track OR functions that define work flow from the physical as well as cognitive point of view and model OR flow to allow efficient multiple OR management scheduling and resource allocation.

### SUMMARY OF THE INVENTION

Presented are systems and methods directed to a monitor medical procedures, including in particular the mental state of medical personnel associated with such procedures. Exemplary embodiments of the present disclosure relate to systems and methods for monitoring medical procedures. Particular embodiments relate to monitoring medical procedures performed in operating room environments through the use of various types of sensors, including for example, wireless electroencephalography (EEG) monitoring systems.

Exemplary embodiments of the present disclosure include a method for non-invasive tracking of OR functions including the mental state of medical personnel. Particular embodiments of the method will allow users to: (i) correlate the steps of the medical procedure with the mental state of the medical personnel in a systematic way; and (ii) build a statistical model that raises an alert when the safety of the procedure should be revisited.

It is important to note that combining the identification of the mental state of the medical personnel and/or patient with tracking the OR function can make the system robust and efficient. In certain embodiments, the system could be used for training purposes and assessment.

While there have been numerous works investigating OR safety issues, it appears that there have been no practical automated solutions implemented according to exemplary embodiments disclosed herein.

It is understood that the issues described above for existing systems and methods are merely exemplary and that other deficiencies can be addressed by the exemplary embodiments disclosed herein. While the existing systems and methods issues described above may appear to be readily addressed, there can be cultural barriers that hinder the ability to address such issues. For example, the medical personnel in the operating room are typically not versed in the arts used to implement solutions (e.g. sensor technologies and computer arts). Similarly, those versed in the arts used to implement solutions are not typically versed in the issues relating to medical procedures.

Embodiments of the present disclosure provide systems and methods for non-invasive tracking of OR functions that can allow the construction of a powerful statistical model of surgery procedures to improve scheduling prior to surgery, as well as and on-the-fly indicators to revise scheduling in real time and reallocate resources when necessary. In certain embodiments, the indicator can be an audible or visual indicator. Exemplary embodiments can track OR functions that define OR work flow, in a noninvasive way, from the physical as well as cognitive point of view, in addition to model OR flow to allow efficient multiple OR management scheduling and resource allocation.

Exemplary embodiments of methods disclosed herein can comprise one or more of the following steps: (i) identify the macro steps in OR flow that are important to multiple OR system management; (ii) associate with each step a noninvasive redundant and robust sensing mechanism that accurately tracks starting and ending times; and (iii) generate a mathematical model of OR management that is amenable to optimum scheduling and resource allocation methods. Diagnostic data from the signal time series can provide a broad variety of information, including for example, time lapse when OR system is not used, time lapse when coordination, staff or equipment resource is lacking, and outliers on anesthesia/surgery time. Exemplary embodiments disclosed herein utilize an agile development procedure that alternates design, testing, and user feedback. In this process, choices made on steps (i) and (ii) are revisited to get improved diagnostic data.

Exemplary embodiments include a medical procedure monitoring system comprising: a computer readable medium comprising a plurality of standards for a medical procedure; and a plurality of sensors comprising an electroencephalography monitoring device. In certain embodiments, each sensor is configured to: detect a parameter of a component used in the medical procedure; and provide an output based on the parameter of the component detected. Particular embodiments include a computer processor configured to: receive the output from each sensor; and compare the output from each sensor to a standard from the plurality of standards for the medical procedure.

In some embodiments, the electroencephalography monitoring device comprises a wireless transmitter. In specific embodiments, the computer processor is configured to compare the output from the electroencephalography monitoring device to a range of a signal standard. In particular embodiments, the system is configured to provide an indicator if the output from the electroencephalography monitoring device is outside of the range of the signal standard. In certain embodiments, the indicator is an indication of drowsiness, and/or cognitive load, and/or personnel dynamics. In some embodiments, the indicator is an audible indicator. In specific embodiments, the indicator is a visual indicator.

In certain embodiments, one of the pluralities of sensors is a component in a surgical tool global positioning system. In particular embodiments, the surgical tool global positioning system comprises: a surgical port comprising a proximal end configured to be located outside a body of a patient and a distal end configured to be located within an internal portion of the body of the patient, and a channel extending between the proximal end and the distal end; a first reference marker positioned at a first fixed location distal to the surgical port; and a camera coupled to the surgical port and configured to capture image data associated with the first reference marker.

Exemplary embodiments include a method of monitoring a medical procedure, the method comprising: monitoring electrical brain activity of a person participating in the medical procedure, where the electrical brain activity is monitored via a electroencephalography monitoring device that provides electroencephalography data; and processing the electroencephalography data to determine if the electroencephalography data is outside an established range. Particular embodiments of the method further comprise providing an indicator if the electroencephalography data is outside the established range. In certain embodiments of the method, the indicator is an indication of drowsiness, and/or cognitive load, and/or personnel dynamics. In some embodiments, the indicator is an audible indicator. In specific embodiments, the indicator is a visual indicator

Particular embodiments of the method further comprise monitoring electrical brain activity of a plurality of persons participating in the medical procedure, where the electrical brain activity of each person is monitored via a electroencephalography monitoring device that provides electroencephalography data for each person; and processing the electroencephalography data for each person to determine if the electroencephalography data for each person is outside an established range. In certain embodiments, the indicator is an indication of personnel dynamics between each of the plurality of persons.

Exemplary embodiments include a method of monitoring medical procedures, the method comprising: identifying a plurality of steps in operating room flow that are critical to multiple operating room system management; associating with each step in the plurality of steps a sensing mechanism that accurately tracks starting and ending times for each step; reconstructing hand motions of a surgeon via a surgical tool global positioning system; monitoring electrical brain activity of a plurality of persons participating in the medical procedure, wherein the electrical brain activity is monitored via a electroencephalography monitoring device that provides electroencephalography data; processing electroencephalography data; and processing the electroencephalography data for each person to determine if the electroencephalography data for each person is outside an established range. Certain embodiments further comprise reconstructing a network of the mental state of the plurality of persons participating in the medical procedure.

The details of one or more embodiments of the invention are set forth in the accompanying drawings and the description below.

Certain terminology is used in the following description are for convenience only and is not limiting. The words “right”, “left”, “lower”, and “upper” designate direction in the drawings to which reference is made. The words “inner”, “outer” refer to directions toward and away from, respectively, the geometric center of the described feature or device. The words “distal” and “proximal” refer to directions taken in context of the item described and, with regard to the instruments herein described, are typically based on the perspective of the surgeon using such instruments. The words “anterior”, “posterior”, “superior”, “inferior”, “medial”, “lateral”, and related words and/or phrases designate preferred positions and orientation in the human body to which reference is made. The terminology includes the above-listed words, derivatives thereof, and words of similar import.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about”, “approximately” or “substantially” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

Exemplary embodiments of the present disclosure are provided in the following drawings. The drawings are merely examples to illustrate the structure of exemplary devices and certain features that may be used singularly or in combination with other features. The invention should not be limited to the examples shown.

FIG. 1 is a schematic diagram of an exemplary embodiment of a system according to the present disclosure;

FIG. 2 is a schematic diagram of an EEG monitoring device according to the present disclosure;

FIG. 3 is a flowchart of steps that can be performed by a computer processor to analyze the output of the EEG monitoring device of FIG. 2.

FIG. 4 is a perspective view of an operating room configured for use with the embodiment of FIG. 1;

FIG. 5 is a table corresponding components configured for monitoring by the embodiment of FIG. 1 and the associated outputs of such components;

FIG. 6 is a table corresponding events or parameters binary outputs of associated sensors configured for use in the embodiment of FIG. 1;

FIG. 7 is a table corresponding procedural events and sensor types configured for monitoring by the embodiment of FIG. 1 and the associated outputs of such components;

FIG. 8 provides illustrations of sensor types for various sensors configured for use with the embodiment of FIG. 1;

FIG. 9 is an illustration of a light sensor configured for use with the embodiment of FIG. 1;

FIG. 10 is an illustration of instrument detection sensor configured for use with the embodiment of FIG. 1;

FIG. 11 is an illustration of a patient entry detection sensor configured for use with the embodiment of FIG. 1;

FIG. 12 is an illustration of a patient transfer detection sensor configured for use with the embodiment of FIG. 1;

FIG. 13 is an illustration of a ventilator status sensor configured for use with the embodiment of FIG. 1;

FIG. 14 is an illustration of a video detection sensor configured for use with the embodiment of FIG. 1;

FIG. 15 is a schematic view of an example system configured for surgical tool global positioning;

FIG. 16 is view of example reference markers of the system of FIG. 1;

FIG. 17 is a schematic diagram of an example tool configured for use with the system of FIG. 15;

FIG. 18 is a schematic view of a tracking element configured for use with the tool of FIG. 17;

FIG. 19 is a schematic diagram of a surgical port of the system of FIG. 15;

FIG. 20 is a schematic of the surgical port of the system of FIG. 15 in a coordinate system;

FIG. 21 is a graph of the trajectory of a reference marker of the system of FIG. 15;

FIG. 22 is a schematic of the rotation of the surgical port of the system of FIG. 15;

FIG. 23 is a schematic of the relationship of the surgical port of the system of FIG. 15 to new image coordinates;

FIG. 24 is a schematic of the initial reconstruction of the coordinates of the surgical port of FIG. 15;

FIG. 25 is a photograph of the camera used to validate data acquired by the system of FIG. 15;

FIG. 26 is a schematic of the camera of FIG. 25.

FIG. 27 is a photograph of reference marks.

FIG. 28 is a photograph of reference marks before and after rotation.

FIG. 29 is a graph of a computational result with different angles.

FIG. 30 illustrates EEG data correlated with medical instrument position data.

FIG. 31 illustrates EEG data for experienced and novice personnel.

FIG. 32 illustrates EEG data for a novice based on a first and third repetition of a procedure.

### DETAILED DESCRIPTION

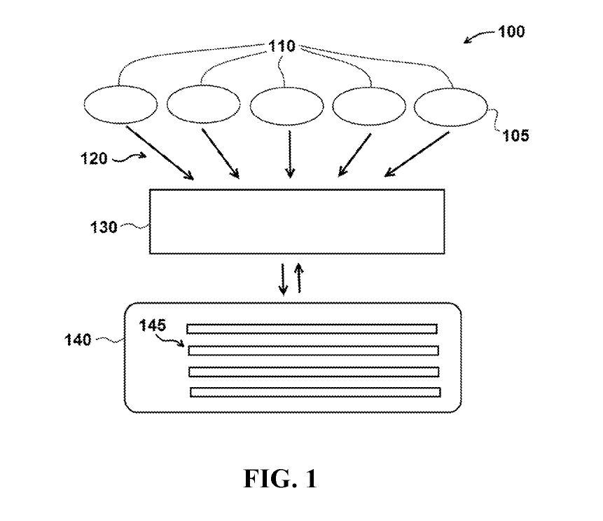

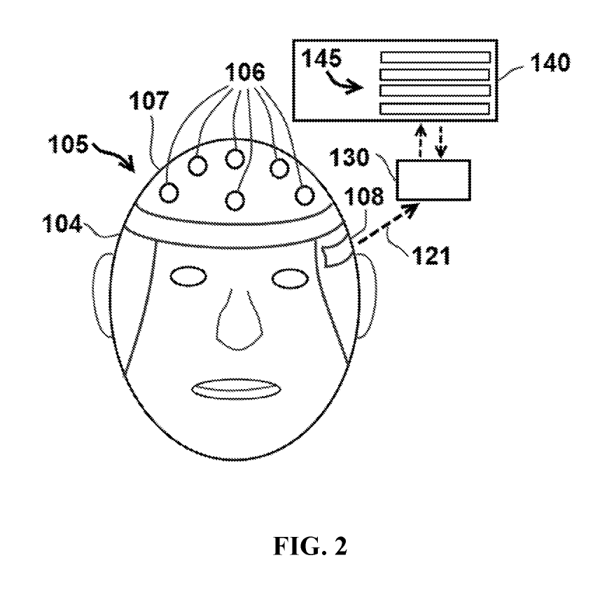

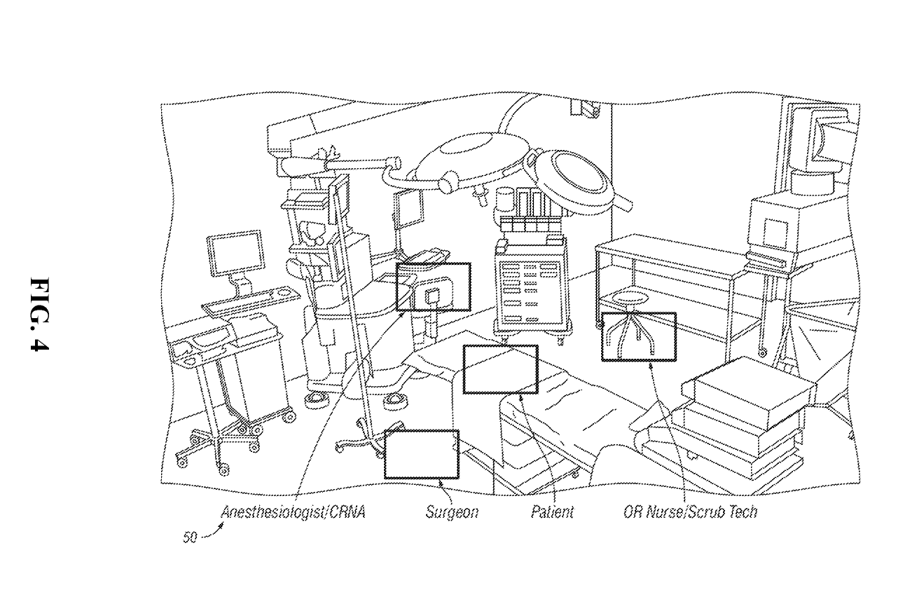

Referring initially to FIGS. 1-4 a schematic of a system 100 configured for medical procedure monitoring is displayed along with an operating room 50 configured for use with system 100. In the embodiment shown, system 100 comprises a plurality of sensors 110 configured to detect a parameter of a component used in a medical procedure (e.g., a procedure performed in operating room 50 shown in FIG. 4). As explained in further detail below, sensors 110 may be configured to detect many different types of parameters, including for example, a component position, operating state, movement, color, or other parameter. As used herein, the term “component” is interpreted broadly to include any device, person or object used in a medical procedure. Examples of components include medical instruments used to directly perform the medical procedure (e.g. scalpels, forceps, catheters, etc.), personnel (patient, surgeon, nurse, anesthesiologist, etc.), and peripheral devices associated with the medical procedure (operating room entry door, draping around patient, etc.) In certain embodiments, the plurality of sensors 110 may comprise an electroencephalography (EEG) monitoring device 105, as shown and described further in FIG. 2.

In exemplary embodiments, sensors 110 can be configured to provide an output 120 based on the parameter of the component detected. In specific embodiments, computer processor 130 is configured to communicate with a computer readable medium 140 comprising a plurality of parameters 145 for a medical procedure. In exemplary embodiments, system 100 may alter the plurality of parameters 145 for the medical procedure (e.g. via a mathematical model) after receiving outputs 120 from each sensor. In particular embodiments, sensors 110 can provide a binary output (based on the detected parameter) to a computer processor 130 configured to receive output 120 from sensors 110.

Referring specifically now to FIG. 2, EEG monitoring device 105 may comprise a plurality of electrodes 106 coupled to a cap 105 that can be worn by a person 104 associated with the medical procedure, including for example, a surgeon, nurse or anesthesiologist. EEG monitoring device 105 may also comprise a wireless transmitter 108 configured to send an EEG signal as output 121 to computer processor 130. In specific embodiments, electrodes 106 may be coupled to a wireless transmitter 108 via one or more wired connections (not shown) in cap 105. In particular embodiments, wireless transmitter 108 can be configured to digitize and amplify analog EEG signals received from electrodes 106. In certain embodiments, the amplified digital EEG signals can be transmitted as output 121 to computer processor 130 via suitable protocols, including for example Bluetooth® wireless transmission.

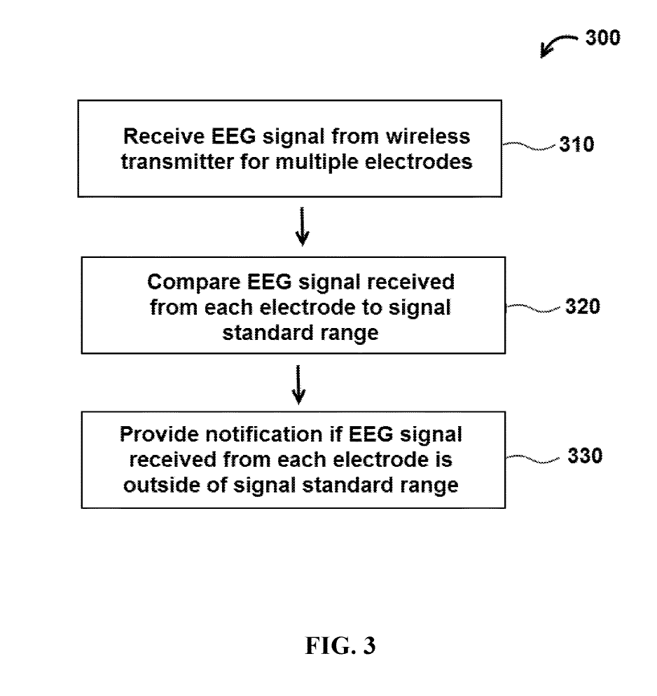

In certain embodiments, computer processor 130 can comprise software that can allow computer processor 130 to analyze EEG signals received from multiple electrodes 106. In particular embodiments, the software can allow computer processor 130 to perform the steps shown in method 300 of FIG. 3. Specifically, allow computer processor 130 can receive the EEG signal from wireless transmitter 108 from multiple electrodes 106. Computer processor 130 can then compare the EEG signal received from each electrode to a signal standard range. If the EEG signal received from each electrode is outside of the signal standard range, a notification can be provided to alert users that the personnel being monitored may have impaired function.

During operation, system 100 can provide important monitoring benefits that can reduce the likelihood of errors in the medical procedure due to the mental state of the personnel associated with the procedure. For example, if the EEG signals received from wireless transmitter 108 are outside a standard range, it can be an indication that the person being monitored is fatigued, stressed, or has been engaged in intense concentration for an extended period of time. In certain embodiments, EEG monitoring of multiple personnel in the operating room can be used to construct a network (e.g. a Bayesian network) of the group mental state based on mental states of each of the individuals.

Medical procedure personnel may initially be reluctant to wear an EEG monitor during medical procedures. However, it is believed that education of the personnel to the potential benefits achieved with such a system can be used to overcome any such reservations. For example, EEG monitoring could be used to establish guidelines for limits on the amount of time medical personnel spend performing medical procedures. This can allow medical personnel to work schedules that permit them to function in an effective manner and reduce the likelihood of mental errors. Such errors could have significant consequences on patients undergoing medical procedures.

Referring back now to FIG. 1, in exemplary embodiments, sensors 110 can be configured to provide an output 120 based on the parameter of the component detected. In specific embodiments, computer processor 130 is configured to communicate with a computer readable medium 140 comprising a plurality of parameters 145 for a medical procedure. In exemplary embodiments, system 100 may alter the plurality of parameters 145 for the medical procedure (e.g. via a mathematical model) after receiving outputs 120 from each sensor. In particular embodiments, sensors 110 can provide a binary output (based on the detected parameter) to a computer processor 130 configured to receive output 120 from sensors 110.

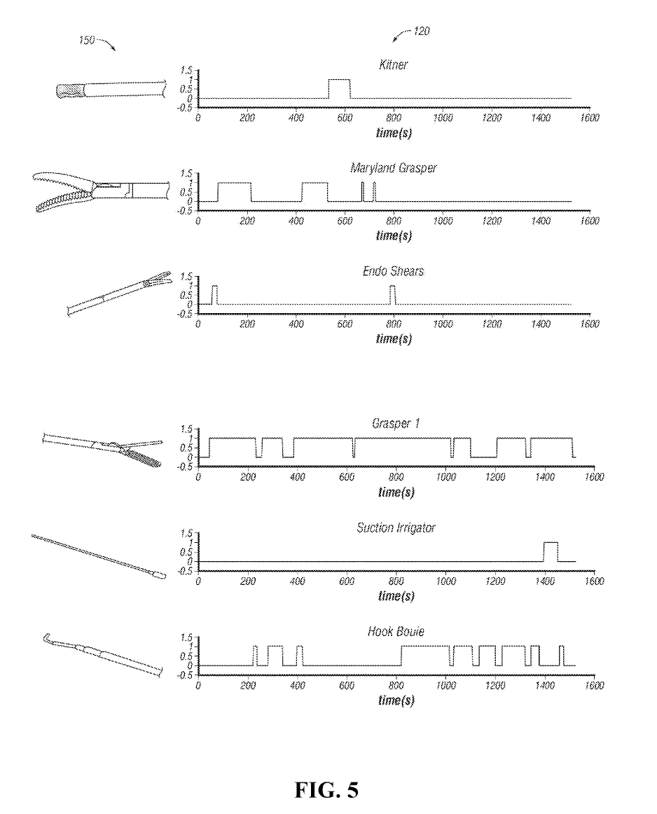

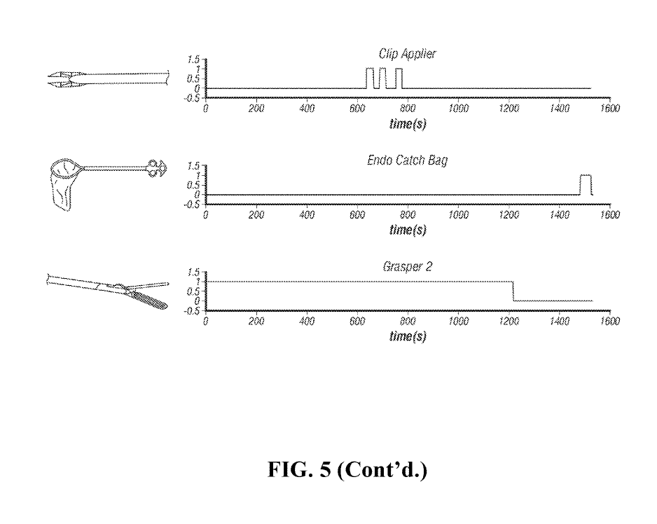

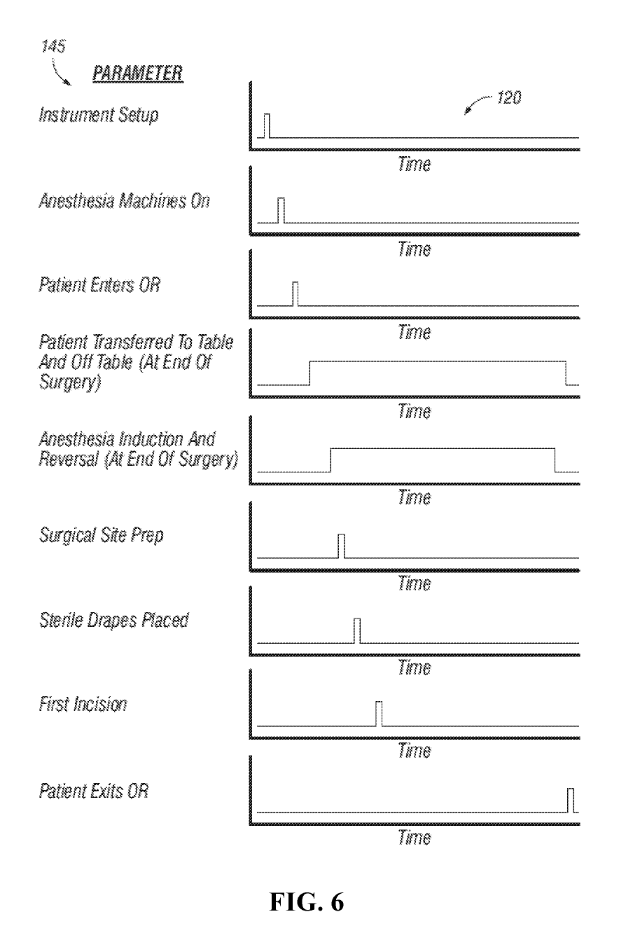

Referring now to FIG. 5, a plurality of components 150 are illustrated beside a corresponding binary output 120 that is indicative of a condition of the component 150. FIG. 6 provides an example of various events or parameters 145 of an exemplary medical procedure and binary outputs 120 of sensors used to detect the events/parameters over time. For example, sensors detecting instrument setup, anesthesia machine operational status and patient entry to the operating room provide a binary discrete output prior to the patient being transferred to the operating table. Sensors detecting the patient's location on the operating room table and anesthesia induction and reversal provide a discrete positive output during the procedure. These two sensor outputs also overlap sensor outputs for surgical site preparation, placement of sterile drapes, and first incision. The final sensor output for the patient exiting the operating room indicates the conclusion of the procedure.

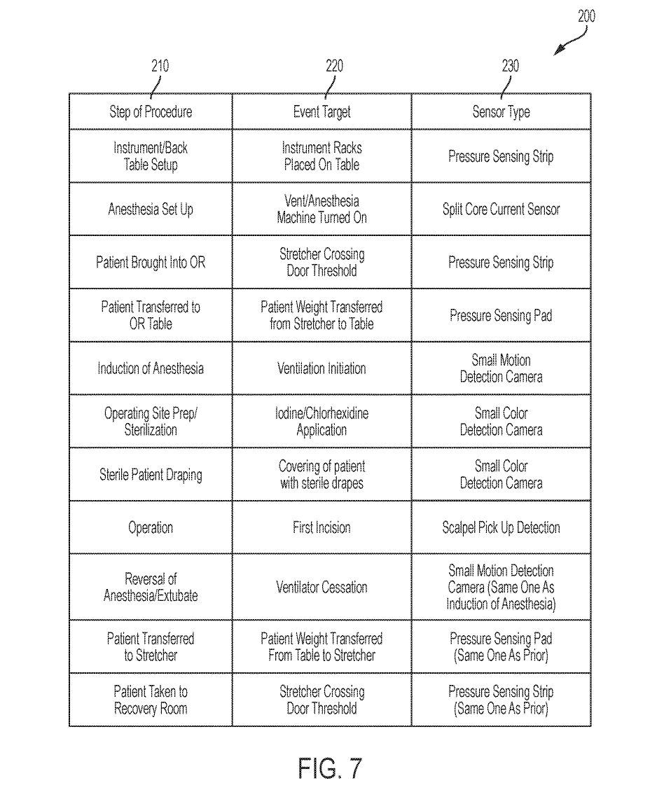

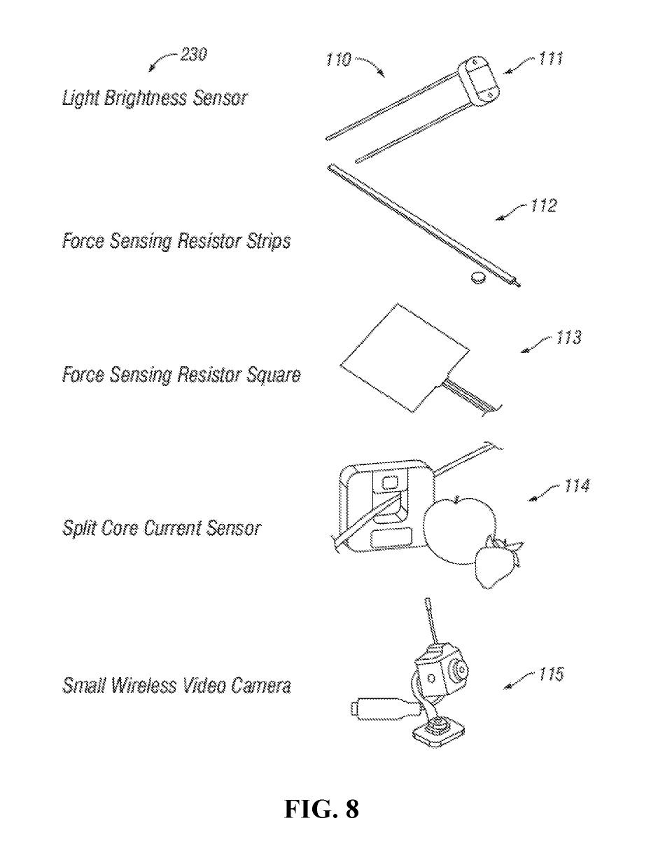

As shown in FIG. 7, a table 200 provides a list of procedure steps 210 (e.g. corresponding to specific standards or parameters 145) in an exemplary medical procedure. In addition, table 200 provides a corresponding list of event targets 220 (e.g. corresponding to outputs 120 for individual sensors 110) and sensor types 230 for various sensors 110. FIG. 8 also provides illustrations of various sensor types (or modalities) 230 for various sensors 110, including a light brightness sensor 111, a force-sensing resistor strip 112, a force-sensing resistor panel 113, a split core current sensor 114, and a video camera 115. It is understood that the provided lists and sensor types are merely exemplary, and that other steps, event targets, and sensor types may be used in other embodiments.

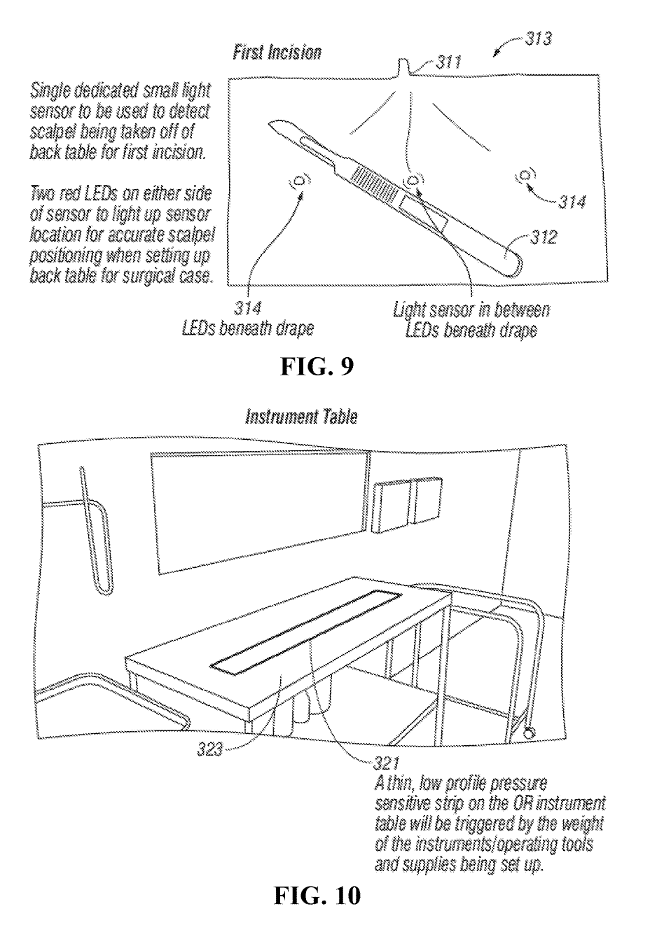

Referring now to FIGS. 9-14, various exemplary embodiments of sensors and their locations are provided. For example, in FIG. 9 a light sensor 311 can be used to detect when a scalpel 312 is removed from a surface 313 (e.g. an instrument table). In a particular embodiment, light sensor 311 may detect light when scalpel 312 is removed from surface 313 to indicate that the instrument is in use. When scalpel 312 is placed back onto the surface 313, light can be blocked from light sensor 311 and sensor 311 can provide an output that scalpel 312 is in a stored location on surface 313 and not in use. In particular embodiments, lights 314 (e.g. LEDs) can be used to indicate the proper position on surface 313 for scalpel 312 to be placed when not in use.

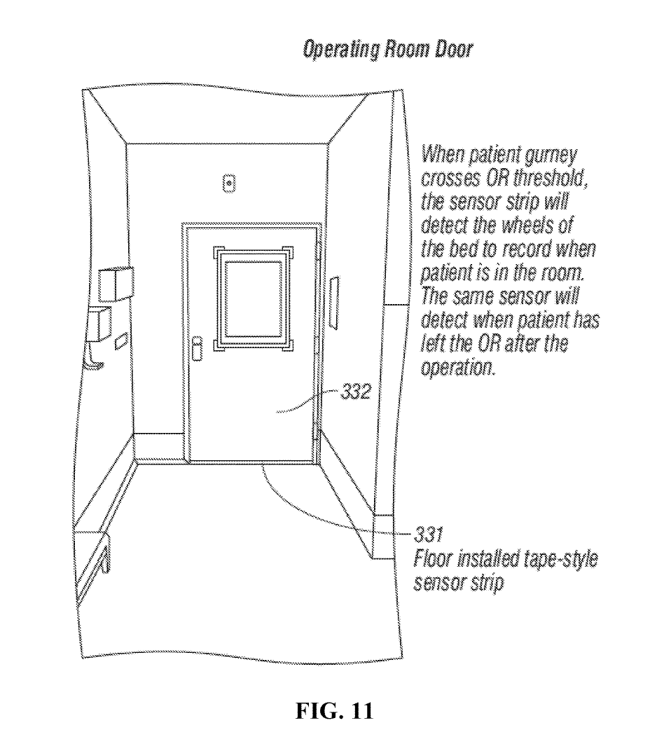

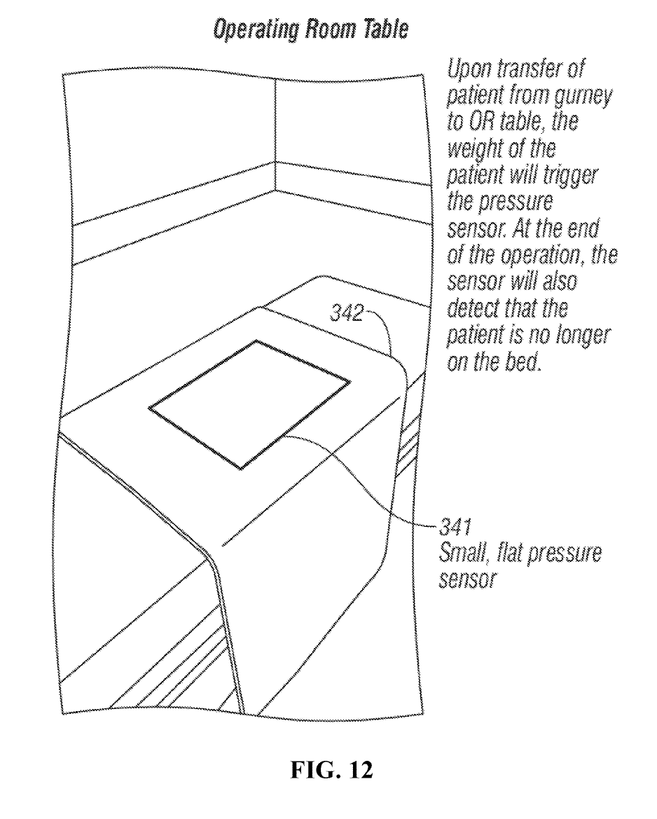

Referring now to FIG. 10, a sensor 321 may be configured as a thin, low profile pressure sensitive strip on surface 323 (e.g. an instrument table). In exemplary embodiments, sensor 321 can detect whether or not an instrument or component is located on surface 323 by the weight of the various instruments or components placed on the surface. As shown in FIG. 11, a sensor 331 may detect when a patient gurney crosses the operating room threshold for entry door 332 to enter or exit the operating room environment. In particular embodiments, sensor 331 may configured as a floor-installed tape-style sensor strip. Referring now to FIG. 12, a sensor 341 can be configured as a pressure sensor (e.g. a force-sensing resistor panel) to detect when a patient has been transferred to or from an operating room table 342.

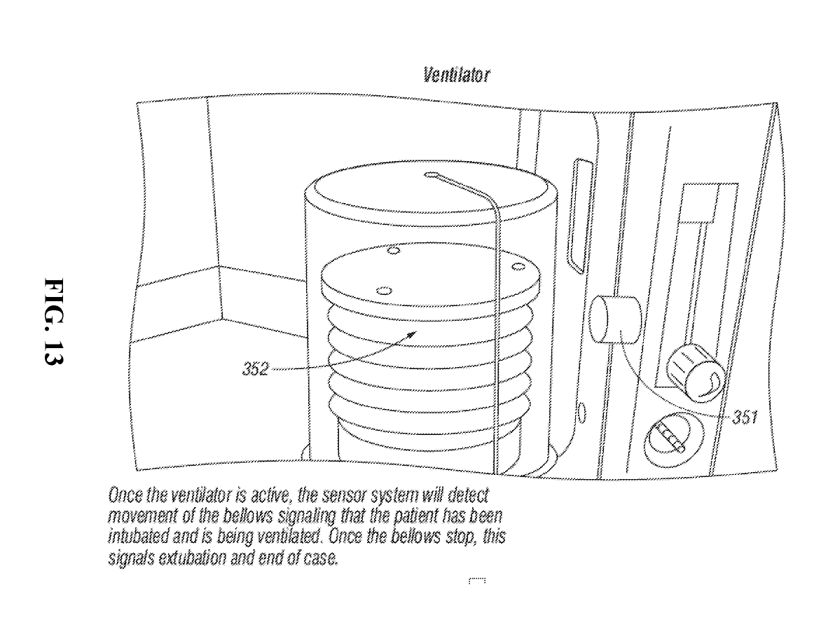

As shown in FIG. 13, a sensor 351 can provide an output to indicate a ventilator 352 is active or inactive. In particular embodiments, sensor 351 may be configured as a video camera configured to detect motion of a bellows in ventilator 352. The active/inactive status of ventilator 352 can be used to determine if the patient is currently intubated and being ventilated. Referring now to FIG. 14, a sensor 361 can be mounted to a ceiling 362 of operating room 50 environment and configured to detect one or more particular colors associated with a specific step of the medical procedure being performed. For example, sensor 361 may be a video camera configured to detect a blue color to indicate that draping has been placed around the patient. In addition, sensor 361 may be configured to detect an orange-brown color to indicate that sterilization solution has been applied to the patient.

The above examples of sensors are not intended to be an exhaustive list of the types of sensors that may be used in exemplary embodiments of the present disclosure. In general, certain sensors in exemplary embodiments may target a specific event, require minimal post-processing, and provide a binary outcome (e.g. “yes/no” for a time event occurrence). Other considerations for sensor selection may include equipment cost and ease of installation (e.g. minimal wiring and no specific sterilization requirements). Still other considerations may include a lack of interference or intrusion with OR equipment and surgical team functions.

Referring back now to FIG. 1, in specific embodiments, computer processor 130 can be configured to communicate with a computer readable medium 130 comprising a plurality of standards 140 for a medical procedure. In exemplary embodiments, system 100 may alter the plurality of standards 140 for the medical procedure (e.g. via a mathematical model) after receiving outputs 120 from each sensor.

In exemplary embodiments, the mathematical model can be developed in conjunction with overall tracking of the OR functions, which provides systematically with no human intervention, an n-uplet (T₁, T₂, . . . , Tₙ) of positive real numbers for each procedure. Tⱼ (j=1 . . . n), represents the time at which each specific targeted event (e.g. those listed in FIG. 5) occurs. The number n of targeted tasks is for example eleven in FIG. 5, and can be adjusted on demand for the surgery application.

Exemplary embodiments of the system disclosed herein are designed to provide robust and accurate data Tⱼ, since each task is tracked by a specific sensor designed for it. (T1, . . . , Tn) represent the time portrait of the surgery procedure, which is a measure of the procedure performance. The average cost of every minute in the OR is approximately $100. This time portrait provides also information on which task interval may take too long.

Exemplary embodiments register the time portrait of each surgery occurring in a given OR, which provides a large data set amenable to standard data mining techniques. For example, clustering these n-uplet in the n dimensional space can rigorously separate standard performance from others with respect to its time portrait. It can also allow computation of the average standard time portrait of a standard procedure and the dispersion around this standard. In addition, it can allow one to classify automatically procedures that are nonstandard into groups and to measure the distance between standard and nonstandard groups to assess economical impact.

One can also look in more details at the relative importance of each events and there interdependency with a principle component analysis. It is also possible to provide the minimum subset of task that provide the same clustering than the original time portrait and therefore target the marker of inefficiency. Furthermore, a database of time portrait can be correlated to the data base of patient outcome after surgery. A main source of information is the National Surgical Quality Improvement Program—http://site.acsnsqip.org/ A rigorous multi-parameter correlation analysis of time portrait with patient outcome can also provide which combination of tasks has maximum impact on quality or failures, such as surgical sight infection.

Embodiments disclosed herein provide a low cost system that does not require new techniques from the surgeon or medical personnel. In addition, the systems and methods are robust and accurate, and can be installed in a standard operating environment. The system also does not present additional risks to patients.

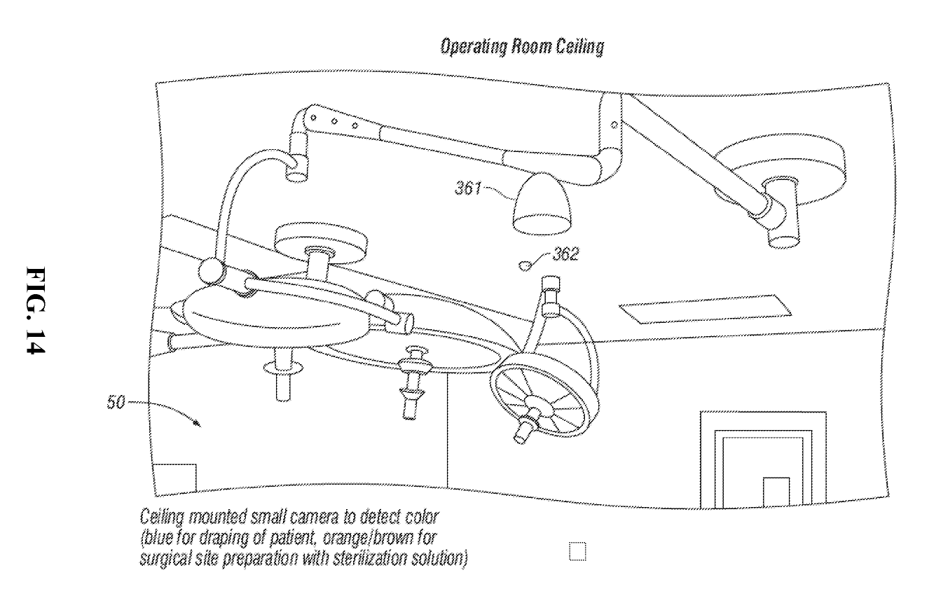

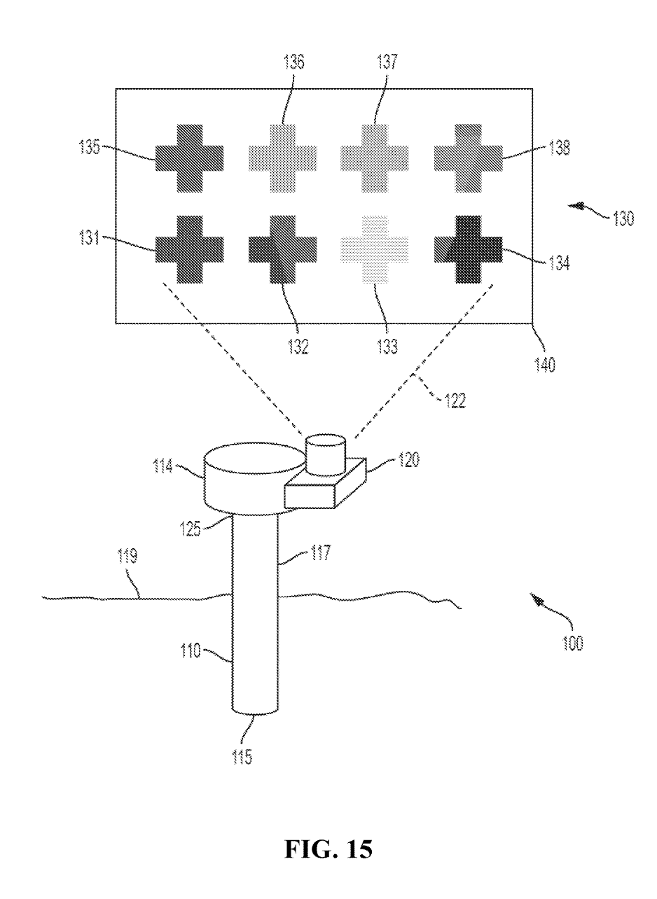

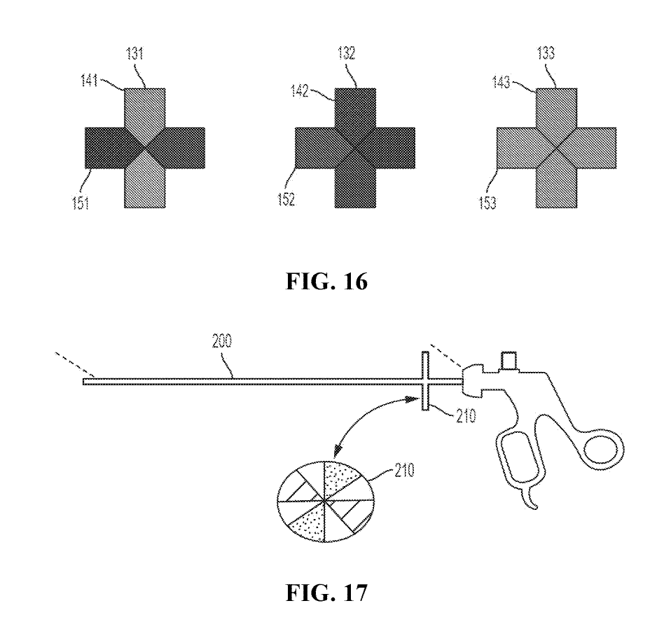

Referring now to FIGS. 15-16, a system 100 configured for surgical tool global positioning is displayed. In the embodiment shown, system 100 comprises a surgical port 110 comprising a proximal end 125 configured to be located outside a body of a patient 119 and a distal end 115 configured to be located within an internal portion of the body of patient 119. In the illustrated embodiment, surgical port 110 comprises a channel 117 extending between proximal end 125 and distal end 115.

In the embodiment of FIGS. 15-16, system 100 further comprises a plurality of reference markers 130 positioned at a first fixed location 140 distal to surgical port 110. In the embodiment shown, the plurality of reference markers 130 comprises individual reference markers 131-138. In particular embodiments, fixed location 140 may be positioned on the ceiling of a room in which surgical port 110 is located, including for example, a ceiling of an operating room.

In addition, the embodiment of system 100 shown comprises a camera 120 coupled to proximal end 125 of surgical port 110. In this embodiment, camera 120 comprises a field of view 122 configured to capture image data associated with one or more reference markers 131-138. As shown in FIG. 16, reference marker 131 may comprise a first segment 141 intersecting with a second segment 151 to form a cross shape. Similarly, reference marker 132 comprises intersecting segments 142 and 152, while reference marker 133 comprises intersecting segments 143 and 153. The remaining reference markers 134-138 can be similarly constructed. It is understood that the geometry, arrangement and number of reference markers shown is merely one example of several different configurations possible in embodiments of the present disclosure.

As explained in more detail below, image data associated with one or more reference markers 131-138 may be used to determine a global position of surgical port 110, as well as a tool inserted into surgical port 110.

Referring now to FIG. 17, a tool 200 is configured for insertion into surgical port 110 (shown in FIG. 15). In this embodiment, a tracking element 210 is coupled to surgical tool 200. As shown in FIG. 17, tracking element 210 is circular in shape and includes a pattern of geometric shapes on one side (e.g. segments of a circle in this embodiment). During use, tool 200 may be inserted into surgical port 110 such that the circular shape and pattern of tracking element 210 can be detected by camera 120. In certain embodiments, tracking element 210 may be configured similar or equivalent to the tool identification marker as disclosed in U.S. patent Ser. No. 14/099,430, incorporated by reference herein. Particular embodiments may also comprise separate cameras for detecting image data associated with tracking element 210 and reference markers 131-138.

In exemplary embodiments, surgical port 110 can be placed into an incision in the body of patient 119 and provide an access point through which surgical instruments may be introduced into an internal surgical site. In certain embodiments, surgical port 110 can include a needle, a cannula, a trocar, or any other style of surgical port known in the art. Surgical port 110 can be composed of a biocompatible material. It is contemplated that the surgical port 110 can be constructed from a disposable material thereby reducing cost and avoiding problems of sterilization and battery change. Surgical port 110 can have a proximal end 125 configured for location on the outside of the patient's body and a distal end 115 sized and configured to extend into the internal portion of the patient's body. Channel 117 can extend through surgical port 110 to provide access to an internal portion of the patient's body such that a surgical tool 200 (e.g. a laparoscope, endoscope or other tool as shown in FIG. 3), can be inserted into the patient's body via channel 117.

Exemplary embodiments of surgical tool tracking system 100 can include a camera 120 mounted to proximal end 125 of surgical port 110. Camera 120 can capture visible spectrum and/or infra-red light or include any other imaging modality suitable for use with surgical procedures. Camera 120 can be configured to capture and store video and/or still images. Camera 120 may also be configured to capture and store audio data. Camera 120 can be configured to capture image data associated with reference markers 130 and tracking element 210 including still and/or video images. Camera 120 may be further configured to capture image data associated with a surgeon performing the medical procedure. For example, camera 120 can capture image data providing surgeon-identifying information such as a surgeon-specific tracking element or marker. An example surgeon-specific marker can include a particular colored glove worn during the medical procedure. The image data associated with the surgeon can also include motion information with respect to surgical tool 106 and/or the surgeon's hand. The motion information can be used to track the motion/path of the surgeon's hands and/or surgical tool 106 during the medical procedure.

In certain exemplary embodiments, camera 120 can be coupled to surgical port 110 via mounting to base 114 of proximal end 125. In other exemplary embodiments, camera 120 can be incorporated with or otherwise integral to base 114. The location of camera 120 with respect to the surgical port 110 can be fixed such that camera 120 can be mounted to or otherwise incorporated into the base 114 at a fixed and set position. In other embodiments, the location of camera 120 can be changed or adjusted with respect to surgical port 110. For example, camera 120 can be mounted to base 114 using an adaptor that controls the position and orientation of camera 120.

In certain embodiments, camera 120 can be mounted to the base 114 such that the optical lens/field of view of camera 120 is directed away from the body of the patient. For example, camera 120 can be mounted to the base 114 such that the optical lens/field of view of camera 120 is provided in a direction of reference markers 131-138, tracking element 210 and/or the surgeon's hand as surgical tool 200 approaches and/or is inserted into surgical port 110. In a further example, camera 120 can be mounted to base 114 such that the optical lens/field of view of camera 120 is both directed away from the body of the patient and in a direction of reference markers 131-138, tracking element 210 and/or the surgeon's hand as surgical tool 200 approaches and/or is inserted into surgical port 110. For example, it is contemplated that the optical lens/field of view of camera 120 can be configured to capture image data of reference markers 131-138, tracking element 210 and/or surgeon's hand as surgical tool 106 approaches and is located within surgical port 110.

In particular embodiments, camera 120 can include a light element for illuminating reference markers 131-138, tracking element 210 and/or the surgeon. For example, light element can include an ultraviolet LED that illuminates a UV sensitive feature on reference markers 131-138 and/or tracking element 210. The use of a non-visible light range should not disturb a surgeon preferring to operate in low light conditions. Use of the a UV sensitive feature on reference markers 131-138 and/or tracking element 210 can also have positive effects on the recognition process because reference markers 131-138 and tracking element 210 will appear to the system a bright and colorful item in the image, thus making it more distinguishable from the background and/or image noise.

In certain embodiments, camera 120 may be capable of operating on a wired or wireless communication network. Camera 120 may be configured to communicate with other devices using the communication network, the other devices including computers, personal data assistants (PDAs), mobile telephones, and mobile computers. For example, tracking system 100 can include a computer system (not shown). Camera 120 can be in communication with the computer system to transmit image data to the computer system for analysis and/or storage. Tracking system 100 may include other components capable of acquiring, storing, and/or processing any form or type of data. Any such component may be coupled to or integrated into base 114 or may be communicatively coupled to tracking system 100 and/or the computer system.

As explained in further detail below, image data obtained by camera 120 and associated with reference markers 131-138 can be used to calculate a global position of laparoscopic tool 200. In the mathematical equations presented herein, it is assumed that the geometry and shape of laparoscopic tool 200 with precise measurement is known. In principle, this information can be provided by the vendor for tool 200. It is also assumed tracking element 210 has a rigid attachment to the tool and is perpendicular to the axis of the tool. The location of the tracking element 210 on the axis is known as shown in FIG. 17.

The motion of laparoscopic tool 200 is channeled by surgical port 110. The motion can be decomposed into: (a) a translation along the main axis of surgical port 110; and (b) a small deviation from the port axis allowed by the difference in diameters between surgical port 110 and tool 200.

The position of the tool 200 in a coordinate system coupled to surgical port 110 can then be determined. If the axis of tool 200 is perfectly aligned to the axis of surgical port 110, the distance from tracking element 210 to surgical port 110 can be computed from the apparent diameter of tracking element 210 in the image data (e.g. video stream). If the port and tool axes are not aligned, tracking element 210 will appear as an ellipse, instead of a circle, in the image data. The axis of the ellipse small diameter and the axis of laparoscopic tool 210 can provide the plan of the rotation.

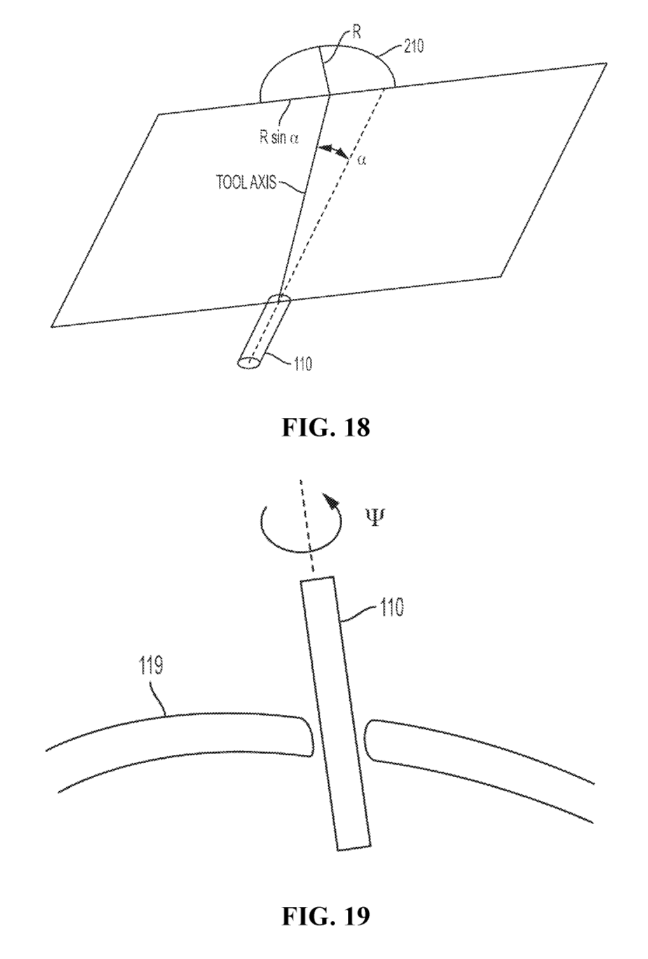

The ratio of the largest diameter of the ellipse to the smallest diameter of the ellipse can provide the angle α via a basic trigonometric formula (see FIG. 18). In practice, a will be small because the diameter of tool 200 is close to that of surgical port 110. For example, a port that is 5 inches in length with a diameter 2 mm larger than the inserted tool will result in a maximum angle α of approximately 1 degree. Based on the geometric constraints and formulas described above, it is possible to localize an end of tool 200 in a coordinate system coupled to surgical port 110.

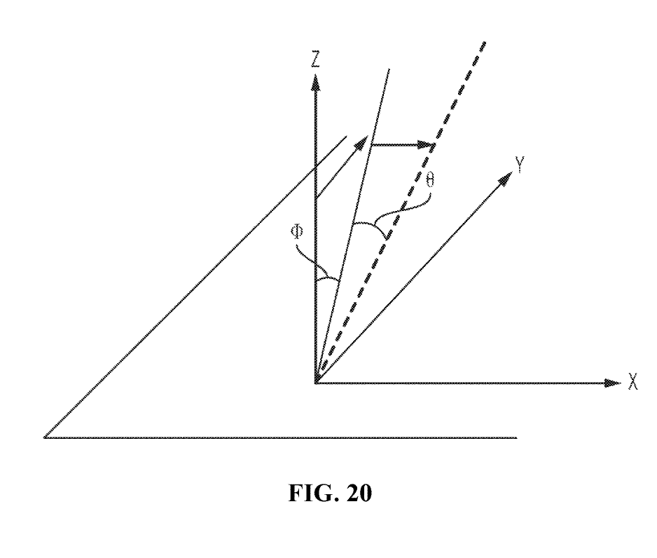

Surgical port 110 can have complex motion in three dimensions. Referring now to FIG. 19, the body of a patient 119 has elasticity, and port 110 can change angle in two independent spatial directions. The motility of patient 119 (e.g. an abdominal wall) can be used by the surgeon to direct the end of tool 200 in the region of interest (ROI). The orientation of the axis of port 110 in the (x, y, z) coordinate system of the operating room corresponds to two unknown angles denoted θ and Φ in FIG. 20. In addition, patient 119 or the support surface (e.g. operating room table) can move during the procedure due to breathing or other movements. Larger movements may correspond to the fact that the surgeon modified the angle of inclination of the support surface to facilitate access to the region of interest. The displacement of location at which port 110 enters patient 119 in three spatial directions is denoted by dx, dy, and dz.

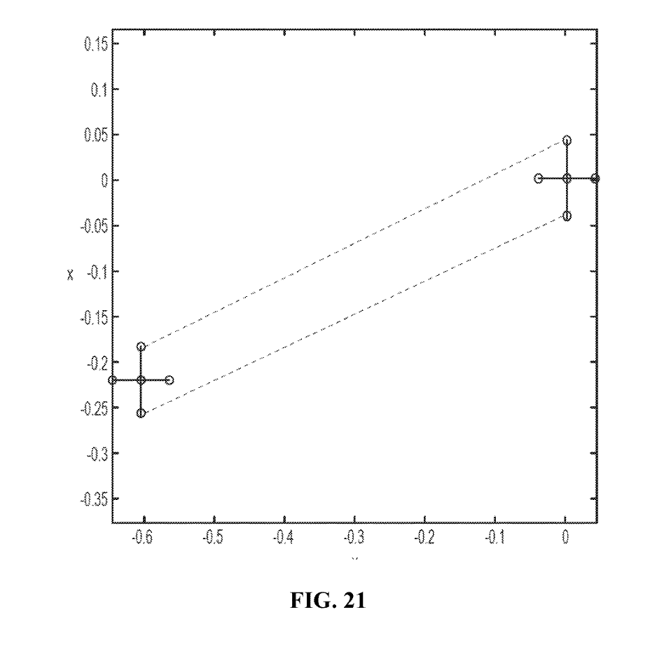

Referring now to FIG. 21, image data (e.g. captured by camera 120) associated with a cross-shaped reference marker (e.g. one of reference markers 131-138) is displayed. From this image data, one can extract the trajectory of five points corresponding to the end points of the intersecting segments and the center of the reference marker. This trajectory corresponds to the motion of surgical port 110. As shown in the sections below entitled “A1 Method” and “A2 Experiment”, mathematical calculations can be performed to determine θ, Φ, dx, dy, and dz. With these values known, one can then reconstruct the spatial trajectory of surgical port 110 in a coordinate system established, for example, in an operating room.

Combining the above parameters and calculations can provide a complete three-dimensional, real-time positioning system for a rigid laparoscopic tool and the tip or end of the tool.

In general, if the tool has mobile parts such as a scissor insert as shown in FIG. 17, one will need to identify the motion of the mobile parts versus the main body of the tool. In many cases, this can be done with a single degree of freedom. One can reconstruct the angle of the opening of the scissor from the image data (e.g. video streaming from an endoscope) to fully reconstruct the position of the tool. Simulated results indicate that accuracy can be obtained on the order of one millimeter for the position of a tool inside an abdominal cavity, and preliminary experimental results confirm the theoretical result.

In certain embodiments, the view angle of camera 120 may be limited and/or obstructed. It may therefore be desirable to include a plurality of reference markers on the ceiling of the operating room. Such a configuration can help to ensure that the system has sufficient input data and can ensure accuracy since the system can use redundant computation. In certain embodiments, the least square fitting method can be used to limit the impact of errors in the pattern recognition of the reference markers. This redundancy may also be used to correct optical distortion when the reference markers are far from the optical axis of the camera. Similarly, in the unlikely event that the surgical port rotates in the plan perpendicular to its axis, one can retrieve the angle of rotation (ψ) as shown in FIG. 19, since there will be multiple reference marker shapes (e.g. crosses of intersecting segments) to reconstruct the additional unknown.

It has been observed that an approximation of the position of a patient abdominal wall can be obtained by virtue of the smart trocars attached to the wall. Provided that one has a three-dimensional reconstruction of the anatomy of the patent in the operating room, one can position the tip of the laparoscopic tool with respect to anatomical structures. The operating room system should then be able to provide information to the surgeon on locations that should not be crossed by the crossed by the laparoscopic tool (e.g. a “secure no fly zone” used in training, but not currently in actual clinical conditions). Similarly, if an optimum access position has been decided during preparation of the operation, the system can guide the surgeon to that optimum maneuver.

Embodiments disclosed herein provide a low cost system that does not require new techniques from the surgeon. In addition, the system is robust and accurate, can be installed in a standard operating environment. The system also does not present additional risks to patients.

It is understood that the methods and mathematical models described in the sections below are exemplary of one embodiment, and that other embodiments are contemplated in this disclosure. For example, while a trocar is referenced in the discussion below, other types of surgical ports may be used in other embodiments.

### A1 Method

For clarity, most of the mathematical presentation below is restricted first to motion in the vertical plane (x,z) that contain trocar. We will discuss briefly second the generalization to three spatial dimension in the (x,y,z) coordinate system of the OR.

Rotation:

Let us consider a rotation of the trocar clockwise in the (x,z) plane. We note this rotation T_{θ}. The trocar has a fixed point that is the center of the rotation. Let is assume the trocar and the marker denoted by the triplet (x₋₁, x₀, x₁)) are in the same vertical plane.

We consider first the direct problem: given θ, what would be the position of the marker in the new image?

In the new coordinate system (\tilde{x},\tilde{y})—see FIG. 15—the coordinate of the marker (x₋₁, x₀, x₁)), is, for j=−1, 0, 1:

\tilde{x}_{J}=cos(θ)(−H tan(θ)+xⱼ), (1)

\tilde{y}_{J}=sin(θ)(−H tan(θ)+xⱼ), (2)

Let us denote Θ_{c} the view of the angle of the camera—see FIG. 16—The physical dimension of the new image frame is (−\tilde{L}, \tilde{L}), on the line \tilde{y}=\tilde{y}_{J}, is:

$$\tilde{L} = tan ( \frac{{Θ}_{C}}{2} ) ( \frac{H}{cos ( θ )} + {\tilde{y}}_{j} ) . \qquad ( 3 )$$

The position of the marker xⱼ in the image (−1, 1) will be

$$\tilde{I} = \frac{{\tilde{x}}_{j}}{\tilde{L}} . \qquad ( 4 )$$

For any landmark of coordinate xⱼ in the initial image, the map

Θ→Ĩⱼ(xⱼ)

for the range of rotation we do consider is bijective. As a matter of fact this map is a strictly decreasing function of θ. The inverse problem consist to solve the non linear set of equation (1) to (4) with for example a Newton scheme.

However we have assumed that the initial position of the trocar in the OR was given. Let us show that this problem can be solved with two landmarks—see FIG. 17. The two unknown are the physical location of the point O at the vertical of the trocar and the ceiling denoted H. For simplicity we will still restrict ourselves to the (x,z) plane. The generalization to 3D is straightforward.

To start we get the coordinate I₀ and I₁ of the landmark x₀ and x₁ in the image. We know also a priori the physical dimension d=x₁−x₀, of our marker.

We have:

$$\begin{matrix}tan ( {θ}_{0} ) = \frac{{x}_{0}}{H} , tan ( {θ}_{1} ) = \frac{{x}_{1}}{H} , tan ( \frac{{θ}_{c}}{2} ) = \frac{L}{H} . \\ and & ( 5 ) \\ {x}_{0} = {I}_{0 } L , {x}_{1} = {I}_{1} L . & ( 6 )\end{matrix}$$

We obtain:

$$H = {d ( ( {I}_{1} - {I}_{0} ) tan ( \frac{{θ}_{c}}{2} ) )}^{- 1} . \\ and$$

$${x}_{0} = {I}_{0} H tan ( \frac{{θ}_{c}}{2} ) , {x}_{1} = {I}_{1} H tan ( \frac{{θ}_{c}}{2} ) .$$

This concludes the reconstruction of the rotation of the trocar by tracking the landmarks on the ceiling.

However the motion of the trocar can be more complex and involve two translations in respectively x and z direction. We will denote dx and dz this displacement and as before θ the rotation.

Translation:

To take into account these two translations, denoted T_{dx} and T_{dz}, the landmark of the initial coordinate xⱼ has for new coordinates

\tilde{x}_{J}=cos(θ)(−H−dz tan(θ)+x₀−dx), (7)

\tilde{y}_{J}=sin(θ)(−H−dz tan(θ)+x₀−dx), (8)

We have now three unknowns that are dx and dy and θ. We need then three landmarks. We need to solve the nonlinear set of equations with the image coordinate I₋₁,I₀,I₁ from these landmarks. We can now use Newton scheme to solve numerically that non linear problem, since we can explicitly compute the Jacobian of the system. So far we have restricted ourselves to two space dimension and we worked with a combination of the three geometric transform:

T_{θ}oT_{dx}oT_{dz}.

A similar reasoning can be applied in three space dimensions. We consider the three d coordinate systems (x,y,z) of the OR. We work with the transformation:

T_{θ}oT_{φ}oT_{dx}oT_{dy}oT_{dz}.

We need then to identify 5 unknowns θ, φ, dx, dy, dz and will need 5 landmarks. We wrote with a matlab code a small simulator based in a cross motif—see FIG. 15. This code applies successively each transformation to the image viewed from the trocar. This simulator helps us compute the sensitivity of the system. Let us assume that the image comes with a resolution of 500 pixels in each dimension. One can show from simulation that an accumulated error of 4 pixel in each spatial direction will result in an error of about 1 mm at the end of the laparoscopic tool. This error is very small indeed because the relative distance from the trocar to the ceiling is much larger than from the trocar to the ROI inside the abdominal cavity.

The exact accuracy of the system needs to be checked with an experiment that will carry various types of uncertainties, from optical defect of the camera, imperfection in focussing, and noise in the image segmentation of the landmark. We expect however to have a fairly robust and accurate result from our design. Next we will present some preliminary experimental results that validate our approach.

A2 Experiment

Our goal here is to validate the quality of the method to reconstruct separately each component of the motion of the trocar, from tracking the landmark on the ceiling.

Rotation:

Let us start with the rotation component in one space dimension. FIG. 18 and FIG. 19 show a rapid prototyping to check that result.

We have set on the ceiling two black crosses that are visible from the digital camera—see FIG. 20. We set first the camera in a flat position, and measure on the wall height of the laser beam projection. We shoot in that position an image of the ceiling.—see FIG. 21 on left. The auto focus option of the camera was turned off. The image of the ceiling is somehow out of focus. We made this image noisy on purpose to get more realistic conditions.

We set the second camera in a position that forms a small angle with the desk as in FIG. 18. We measure on the wall the new position of the laser beam projection. From these two measures on the wall, we get the angle α with an accuracy of about 0.5°. We shoot in that new position an image of the ceiling—see FIG. 21 on right.

We observe indeed the displacement of the markers due to the change of orientation of the camera.

We apply then our algorithm to reconstruct the angle α from these two images: first we compute the coordinate of the three points A, B, and C using the graphic interference of the GIMP2 software. An automatic image segmentation will be actually more accurate.

Second we map the transformation we defined earlier

θ→Ĩⱼ(xⱼ)

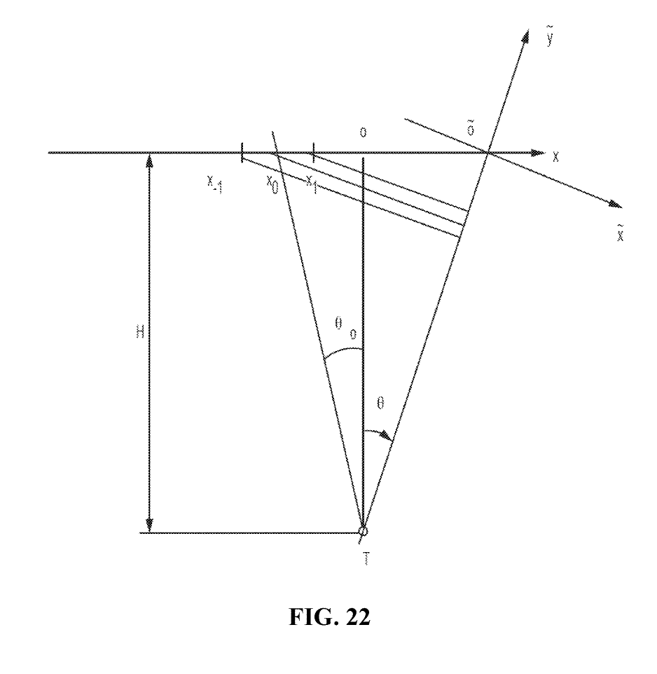

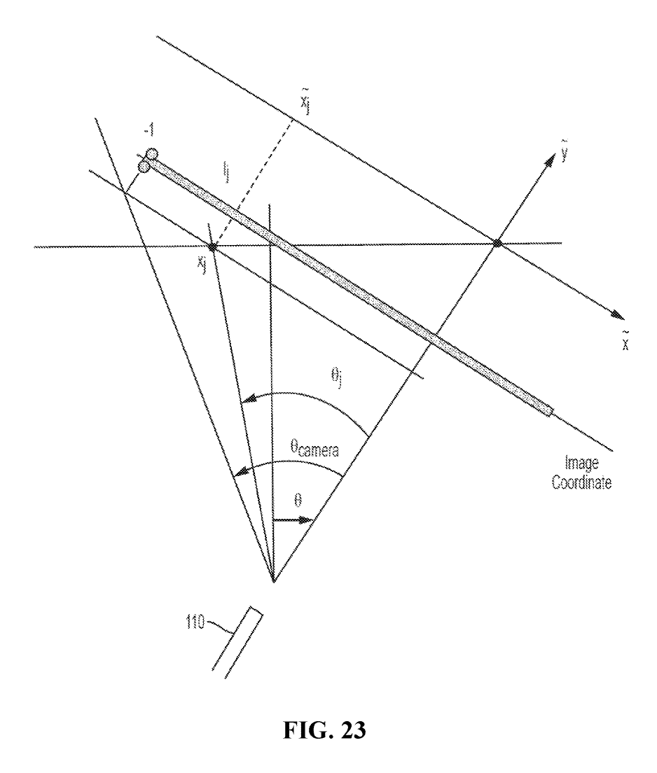

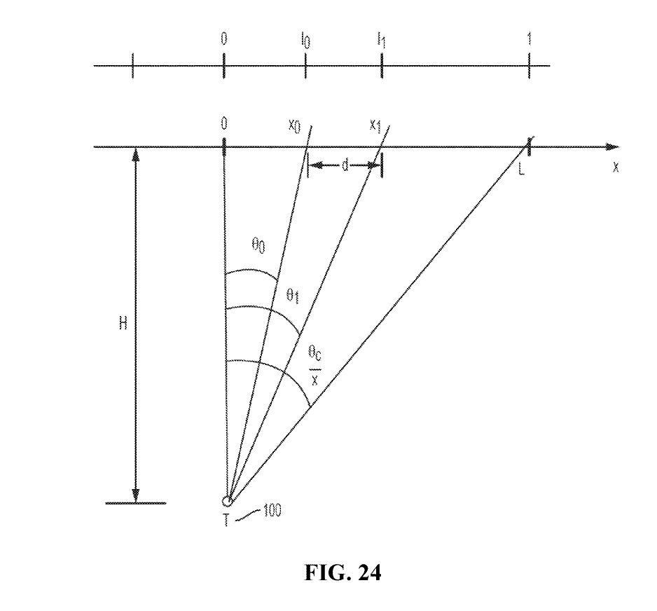

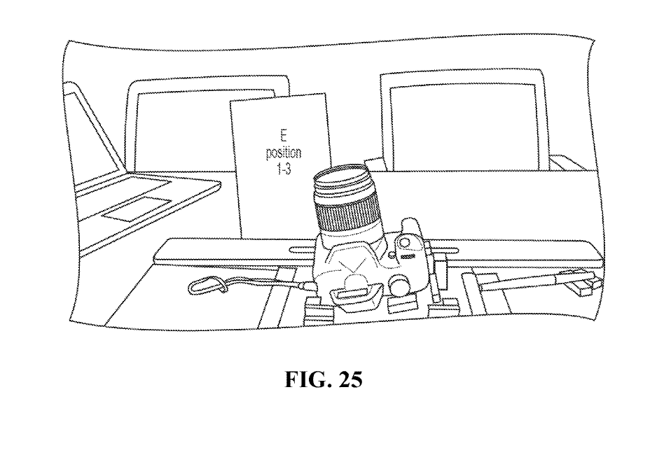

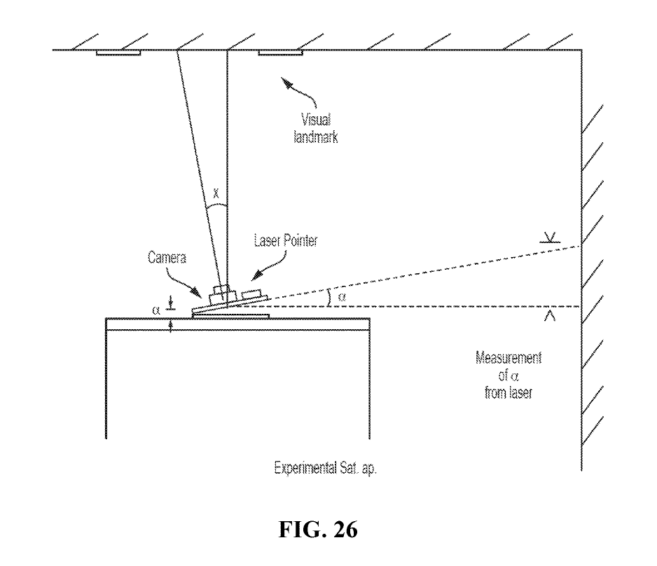

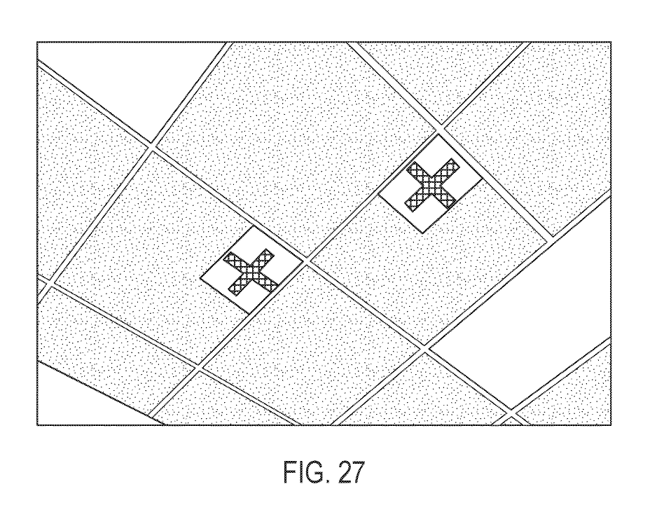

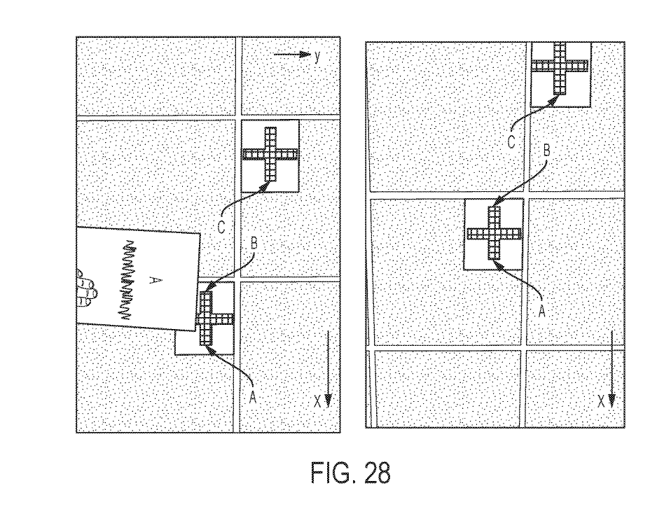

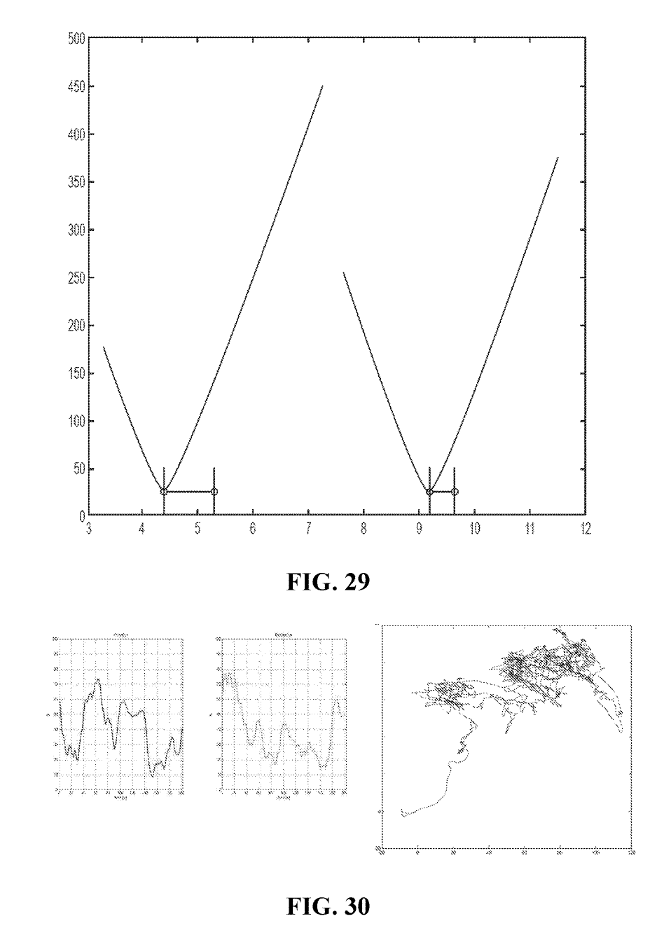

and look for the angle that minimizes the matching between the compound coordinate of the point A, B and C after rotation, in the L2 norm—FIG. 22. Our results are for α=5.3° and α=9.6°. Our algorithm based on computer vision gives: α=4.4° and α=9.2°. We did this experiment several times, and observed a good reliability of the method.

In other words we get an error of less than a degree on the trocar position. This may represent an error on the lateral position of the tip of a laparoscopic tool of the order of 3 mm for a ROI with a 20 cm depth from the abdominal wall.

Translation:

Next let us consider a different displacement of the trocar that can be for example resulting from a patient breathing.

We have run a similar experiment to check the accuracy of a displacement of the “trocar” in the vertical direction z toward the ceiling. Here the camera stays flat, and we change the thickness of the support, to increase the height of a few centimeters. Let's denote δz the increase in thickness of the support. For δz=2 cm we get from our computer vision algorithm a value of δz=1.62 cm. Similarly for δz=3 cm we get from our computer vision algorithm a computed value of δz=3.23 cm. Overall the error on the vertical displacement is less than 4 mm. We suspect that we can improve much that result by using landmarks separated by larger distances.

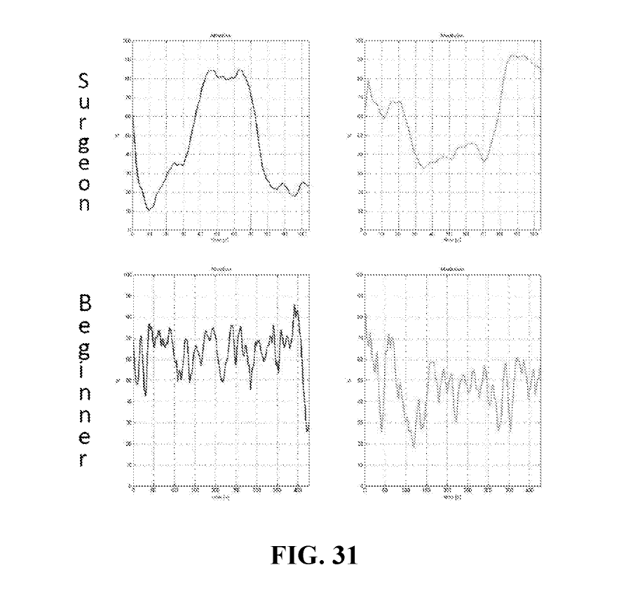

Referring now to FIG. 30, a correlation is shown between EEG data measured and the position of a tool as measured by a global tool positioning system. As noted in the figure, the two visible clusters of activity measured by the positioning system correlate with the higher percentage of attention and lower percentage of meditation (at approximately 30 and 90 seconds).

Referring now to FIG. 31, a comparison is shown of EEG data measured for an experienced surgeon and a beginner when manipulating a medical instrument to perform a task. As shown in the figure, the surgeon is able to relax before and after the exercise due to his familiarity. He is also able to stay focused on his task during all of the exercise. In contras the beginner is less attentive (60% of attention in average), has some lack of attention, and cannot relax before and after the exercise.

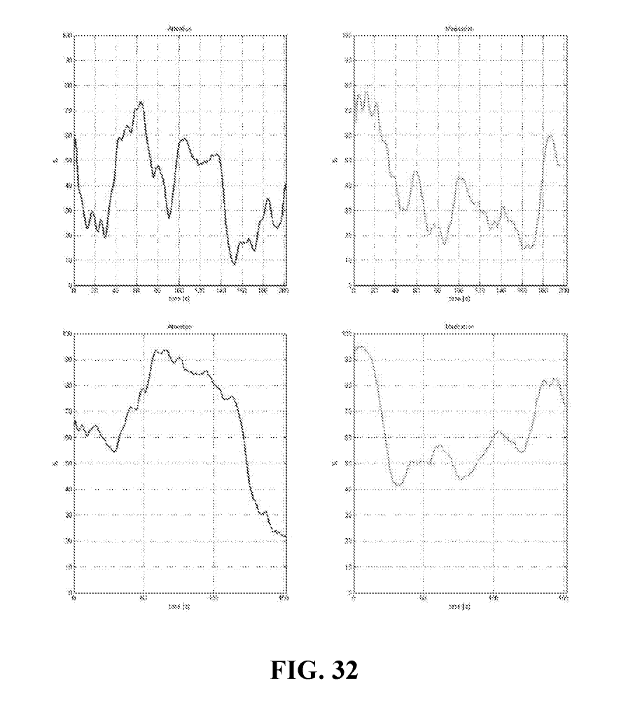

Referring now to FIG. 32, a comparison is made of a novice performing an exercise for the first time (top) and the third time (bottom). After only three repetitions, the EEG data for the novice looks similar to that of the experienced surgeon. However, the novice still required 90 seconds to perform the task while the surgeon completed the task in only 40 seconds.

It is understood that the methods and mathematical models described in the sections below are exemplary of one embodiment, and that other embodiments are contemplated in this disclosure.

While the foregoing description and drawings represent examples of the present invention, it will be understood that various additions, modifications, combinations and/or substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed examples are, therefore, to be considered in all respects as illustrative and not restrictive, the scope of the invention being indicated by the appended claims and not limited to the foregoing description.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention, as defined by the following claims.

### REFERENCES

The contents of the following references are incorporated by reference herein:
[1] R. Marjamaa, A. Vakkuri, and O. Kirvel, “Operating room management: why, how and by whom?,” Acta Anaesthesiologica Scandinavica, vol. 52, no. 5, pp. 596-600, 2008.
[2] B. T. Denton, A. J. Miller, H. J. Balasubramanian, T. R. Huschka, Optimal allocation of Surgery Blocks to Operating Rooms under Uncertainty, Operation Research 58, 2010, pp 802-816
[3] I. Ozkarahan, Allocation of Surgeries to Operating Rooms by Goal Programing, Journal of Medical Systems, Vol 24, No 6, 2000.
[4] D. N. Pham and A. Klinkert, Surgical case scheduling as a generalized job shop scheduling problem, European Journal of Operational Research, Vol 185, Issue 3, 2008, pp 1011-1025.
[5] F. Dexter, A. Macaro, L. O'Neill, Scheduling surgical cases into overflow block time-computer simulation of the effects of scheduling strategies on operating room labor costs, Anesth Analg 2000, 90 (4) 980-8.
[6] Choi S, Wilhelm W E., On capacity allocation for operating rooms. Computers and Operations Research 44:174-184, 2014
[7] Sperandio F, Gomes C, Borges J, Brito A C, Almada-Lobo B. An intelligent decision support system for the operating theater: a case study. IEEE Transactions on Automation Science and Engineering 11:265-273, 2014
[8] Banditori C, Cappanera P, Visintin F., A combined optimization-simulation approach to the master surgical scheduling problem. IMA Journal of Management Mathematics 24:155-187, 2013
[9] Avery D M III, Matullo K S., The efficiency of a dedicated staff on operating room turnover time in hand surgery. The Journal of Hand Surgery 39:108-110, 2014
[10] Kodali B S, Kim K D, Flanagan H, Ehrenfeld J M, Urman R D. Variability of subspecialty-specific anesthesia-controlled times at two academic institutions. Journal of Medical Systems 38:11, 2014
[11] Meskens N, Duvivier D, Hanset A. Multi-objective operating room scheduling considering desiderata of the surgical team. Decision Support Systems 55:650-659, 2013 References on Impact of OR Management on finance, staffing and surgical outcome:
[12] Dexter F, et al. Use of Operating Room Information System Data to Predict the Impact of Reducing Turnover Times on Staffing Costs. Anesth Analy 2003; 97:1119-26.
[13] Abouleish A, et al. Labor Costs Incurred by Anesthesiology Groups Because of Operating Rooms Not Being Allocated and Cases Not Being Scheduled Maximize Operating Room Efficiency. Anesth Analg 2003; 96: 1109-13.
[14] Macario, Alex. Are You Hospital Operating Rooms “Efficient”? Anesthesiology 2006; 105:237-40.
[15] Strum D P, Vargas L G, May J H, Bashein G. Surgical suite utilization and capacity planning: a minimal cost analysis model. J Med Syst 1997; 21:309-22.
[16] Alarcon A, Berguer R., A comparison of operating room crowding between open and laparoscopic operations. Surgical Endoscopy 1996; 10(9):916-19.
[17] Papaconstantinou H T, Smythe W R, Reznik S I, Sibbitt S, Wehbe-Janek H. Surgical safety checklist and operating room efficiency: results from a large multispecialty tertiary care hospital. The American Journal of Surgery 206:853-860, 2013
[18] Radcliff K E, Rasouli M R, Neusner A, Kepler C K, Albert T J, Rihn J A, Hilibrand
A S, Vaccaro A R. Preoperative delay of more than 1 hour increases the risk of surgical site infection. Spine 38:1318-1323, 2013
[19] Schuster M, Pezzella M, Taube C, Bialas E, Diemer M, Bauer M. Delays in starting morning operating lists: an analysis of more than 20,000 cases in 22 German hospitals. Deutsches Arteblatt International 110:237-243, 2013
[20] Warner C J, Walsh D B, Horvath A J, Walsh T R, Herrick D P, Prentiss S J, Powell R J. Lean principles optimize on-time vascular surgery operating room starts and decrease resident work hours. Journal of Vascular Surgery 58:1417-1422, 2013
[21] Carey K, Burgess J F Jr, Young G J. Hospital competition and financial performance: the effects of ambulatory surgery centers. Health Economics 20:571-581, 2011
[22] Fry D E, Pine M, Jones B L, Meimban R J., The impact of ineffective and inefficient care on the excess costs of elective surgical procedures. Journal of American College of Surgeons 212:779-786, 2011
[23] Helmreich R, Davies J. 3 Human Factors in the Operating Room: Interpersonal Determinants of Safety, Efficiency, and Morale. Balliere's Clinical Anaesthesiology. Vol 10, Issue 2 1996, pp 277-295.
[24] Siciliani, L., Hurst, J., Tackling excessive waiting times for elective surgery: a comparative analysis of policies in 12 OECD countries. Health Policy 2005; 72:201-215.
[25] Dexter F, Willemsen-Dunlap A, Lee J. Operating Room Managerial Decision-Making on the Day of Surgery with and Without Computer Recommendations and Status Displays. Anesth Analg 2007; 105:419-29.
[26] Agarwal S, Joshi A, Finin T, Yesha Y., A Pervasive Computing System for the Operating Room of the Future. Mobile Networks and Applications. 2007; 12:215-28.
[27] A. Doryab, and J. E. Bardram, “Designing activity-aware recommender systems for operating rooms,” in Proceedings of the 2011 Workshop on Contextawareness in Retrieval and Recommendation (CaRR '11), New York, N.Y., USA, 2011, pp. 43-46.
[28] A. Doryab, J. Togelius, and J. Bardram, “Activity-aware recommendation for collaborative work in operating rooms,” in Proceedings of the 2012 ACM international conference on Intelligent User Interfaces (IUI '12), New York, N.Y., USA, 2012, pp. 301-304.
[29] Bouarfa, P. P. Jonker, J. Dankelman, Discovery of high-level tasks in the operating room, Journal of Biomedical Informatics, In Press, DOI: 10.1016/j.jbi.2010.01.004.
Neumuth T, StrauB G, Meixensberger J, Lemke H, Burgert O. Acquisition of Process Descriptions from Surgical Interventions. Lecture notes in computer science. 2006; 4080:602-11.
[30] T. Blum, N. Padoy, H. Feussner, and N. Navab, “Modeling and online recognition of surgical phases using Hidden Markov Models,” Med Image Comput Comput Assist Interv, vol. 11, no. Pt 2, pp. 627-35, 2008.
[31] N. Padoy, T. Blum, S.-A. Ahmadi, H. Feussner, M.-O. Berger, and N. Navab, “Statistical modeling and recognition of surgical workflow,” Medical Image Analysis, vol. 16, no. 3, pp. 632-641, 4//, 2012.
[32] T. Neumuth, P. Jannin, J. Schlomberg, J. Meixensberger, P. Wiedemann, and O. Burgert, “Analysis of surgical intervention populations using generic surgical process models,” Int J Comput Assist Radiol Surg, vol. 6, no. 1, pp. 59-71, January, 2011.
[33] D. Neumuth, F. Loebe, H. Herre, and T. Neumuth, “Modeling surgical processes: a four-level translational approach,” Artif Intell Med, 3, pp. 147-61, Netherlands: 2010 Elsevier B. V, 2011.
[34] Yan Xiao, Stephen Schimpff, Colin Mackenzie, Ronald Merrell, Eileen Entin, Roger Voigt, and Bruce Jarrell, Video Technology to Advance Safety in the Operating Room and Perioperative Environment, Surgical Innovation, March 2007 14: 52-61.
[35] M. Allan, S. Ourselin, S. Thompson, D. J. Hawkes, J. Kelly and D. Stoyanov, Toward detection and localization of instruments in minimally invasive surgery, IEEE Transactions on Bio-medical Engineering, April 2013.
[36] Dutkiewicz P, Kielczewski M, Kowalski M. Visual tracking of surgical tools for laparoscopic surgery. Paper presented at: Robot Motion and Control, 2004. RoMoCo '04. Proceedings of the Fourth International Workshop on; 17-20 Jun. 2004, 2004.
[37] Climent J, Mares P. Real-time tracking system for assisted surgical operations. Latin America Transactions, IEEE (Revista IEEE America Latina). 2003; 1(1):8-14.
[38] Dutkiewicz P, Kietczewski M, Kowalski M, Wroblewski W. Experimental verification of visual tracking of surgical tools. Paper presented at: Robot Motion and Control, 2005. RoMoCo '05. Proceedings of the Fifth International Workshop on; 23-25 Jun. 2005, 2005.
[39] Staub C, Lenz C, Panin G, Knoll A, Bauernschmitt R. Contour-based surgical instrument tracking supported by kinematic prediction. Paper presented at: Biomedical Robotics and Biomechatronics (BioRob), 2010 3rd IEEE RAS and EMBS International Conference on; 26-29 Sep. 2010, 2010.
[40] Blasinski H, Nishikawa A, Miyazaki F. The application of adaptive filters for motion prediction in visually tracked laparoscopic surgery. Paper presented at: Robotics and Biomimetics, 2007. ROBIO 2007. IEEE International Conference on; 15-18 Dec. 2007, 2007.
[41] Payandeh S, Xiaoli Z, Li A. Application of imaging to the laparoscopic surgery. Paper presented at: Computational Intelligence in Robotics and Automation, 2001. Proceedings 2001 IEEE International Symposium on; 2001, 2001.
Society of American Gastrointestinal and Endoscopic Surgeons, http://www.sages.org/
[42] J. R. Colombo, Jr., G. P. Haber, M. Rubinstein, and I. S. Gill, “Laparoscopic surgery in urological oncology: brief overview,” Int Braz J Urol, 5, pp. 504-12, Brazil, 2006.
[43] D. Herron, M. Gagner, T. Kenyon, and L. Swanstrm, “The minimally invasive surgical suite enters the 21st century,” Surgical Endoscopy, vol. 15, no. 4, pp. 415-422, 2001.
[44] Liu C C, Chang C H, Su M C, Chu H T, Hung S H, Wong J M, et al. RFID-initiated workflow control to facilitate patient safety and utilization efficiency in operation theater. Comput Methods Programs Biomed. 2011; 104(3):435-42.
[45] J. E. Bardram, A. Doryab, R. M. Jensen, P. M. Lange, K. L. G. Nielsen, and S. T. Petersen, “Phase recognition during surgical procedures using embedded and body-worn sensors.” pp. 45-53.
[46] C. C. Liu, C. H. Chang, M. C. Su, H. T. Chu, S. H. Hung, J. M. Wong, and P. C. Wang, “RFID-initiated workflow control to facilitate patient safety and utilization efficiency in operation theater,” Comput Methods Programs Biomed, 3, pp. 435-42, Ireland: A 2010 Elsevier Ireland Ltd, 2011.
[47] M. Kranzfelder, A. Schneider, G. Blahusch, H. Schaaf, and H. Feussner, “Feasibility of opto-electronic surgical instrument identification,” Minim Invasive Ther Allied Technol, 5, pp. 253-8, England, 2009.
[48] Tatar F, Mollinger J, Bossche A. Ultrasound system for measuring position and orientation of laparoscopic surgery tools. Paper presented at: Sensors, 2003. Proceedings of IEEE; 22-24 Oct. 2003, 2003.
[49] Tatar F, Mollinger J R, Bastemeijer J, Bossche A. Time of flight technique used for measuring position and orientation of laparoscopic surgery tools. Paper presented at: Sensors, 2004. Proceedings of IEEE; 24-27 Oct. 2004, 2004.
[50] Nakamoto M, Nakada K, Sato Y, Konishi K, Hashizume M, Tamura S. Intraoperative Magnetic Tracker Calibration Using a Magneto-Optic Hybrid Tracker for 3-D Ultrasound-Based Navigation in Laparoscopic Surgery. Medical Imaging, IEEE Transactions on. 2008; 27(2):255-270.
[51] B. Estebanez, P. del Saz-Orozco, I. Rivas, E. Bauzano, V. F. Muoz and I. Garcia-Morales, Maneuvers recognition in laparoscopic surgery: Artificial Neural Network and hidden Markov model approaches, 4th IEEE RAS & EMBS International Conference on Biomedical Robotics and Biomechatronics, pp 1164-1169, 24-27 Jun. 2012.
[52] J. Stoll, H. Ren and P. E. Dupont, Passive Markers for Tracking Surgical Instruments in Real-Time 3-D Ultrasound Imaging, IEEE Transactions on Medical Imaging, Volume 31, Issue 3, pp 563-575, March 2012.
[53] Voros S, Orvain E, Cinquin P, Long J A. Automatic detection of instruments in laparoscopic images: a first step towards high level command of robotized endoscopic holders. Paper presented at: Biomedical Robotics and Biomechatronics, 2006. BioRob 2006. The First IEEE/RAS-EMBS International Conference on; 20-22 Feb. 2006, 2006.
[54]J. S.-C. Yuan, A general photogrammetric method for determining object position and orientation, IEEE Transactions on Robotics and Automation, Volume 5, Issue 2, pp 129-142, April 1989.
[55] G. Toti, M Garbey, V. Sherman, B. Bass and B. Dunkin, Smart Trocar for Automatic
Tool Recognition in Laparoscopic Intervention, to appear in Surgical Innovation—SAGE Journals.
[56] Pressure sensing strip to detect when table's instruments are placed on top http://www.pololu.com/product/1697
[57] Split core current sensor to detect when instruments turned on https://www.google.com/shopping/suppliers/search?source=cunit&group=Sensors+and+Transducers&gclid=CPPwn8i61rwCFaZAMgod1hcASA&q=split+core+current+sensor&oq=current+sensor
[58] Pressure sensing pad to detect patient weight on OR table http://www.pololu.com/product/1645
[59] E. Durucan and T. Ebrahimi, Change Detection and Background Extraction by Linear Algebra, Invited Paper Proceedings of the IEEE, Vol 89, No 10, October 2001.

What is claimed is:

1. A medical procedure monitoring system comprising:
a computer readable medium comprising a plurality of standards for a medical procedure;
a plurality of sensors comprising an electroencephalography monitoring device, wherein each sensor is configured to:
detect a parameter of a component used in the medical procedure;
provide an output based on the parameter of the component detected; and
a computer processor configured to:
receive the output from each sensor; and
compare the output from each sensor to a standard from the plurality of standards for the medical procedure.

2. The medical procedure monitoring system of claim 1 wherein the electroencephalography monitoring device comprises a wireless transmitter.

3. The medical procedure monitoring system of claim 1 wherein the computer processor is configured to compare the output from the electroencephalography monitoring device to a range of a signal standard.

4. The medical procedure monitoring system of claim 3 wherein the system is configured to provide an indicator if the output from the electroencephalography monitoring device is outside of the range of the signal standard.

5. The medical procedure monitoring system of claim 4 wherein the indicator is an indication of drowsiness.

6. The medical procedure monitoring system of claim 4 wherein the indicator is an indication of cognitive load.

7. The medical procedure monitoring system of claim 4 wherein the indicator is an indication of personnel dynamics.

8. The medical procedure monitoring system of claim 4 wherein the indicator is an audible indicator.

9. The medical procedure monitoring system of claim 4 wherein the indicator is a visual indicator.

10. The medical procedure monitoring system of claim 1 wherein one of the plurality of sensors is a component in a surgical tool global positioning system.

11. The medical procedure monitoring system of claim 10 wherein the surgical tool global positioning system comprises:
a surgical port comprising a proximal end configured to be located outside a body of a patient and a distal end configured to be located within an internal portion of the body of the patient, and a channel extending between the proximal end and the distal end;
a first reference marker positioned at a first fixed location distal to the surgical port; and
a camera coupled to the surgical port and configured to capture image data associated with the first reference marker.

12. A method of monitoring a medical procedure, the method comprising:
monitoring electrical brain activity of a person participating in the medical procedure, wherein the electrical brain activity is monitored via a electroencephalography monitoring device that provides electroencephalography data; and
processing the electroencephalography data to determine if the electroencephalography data is outside an established range.

13. The method of claim 12 further comprising providing an indicator if the electroencephalography data is outside the established range.

14. The method of claim 13 wherein the indicator is an indication of drowsiness.

15. The medical procedure monitoring system of claim 13 wherein the indicator is an indication of cognitive load.

16. The medical procedure monitoring system of claim 4 wherein the indicator is an audible indicator.

17. The medical procedure monitoring system of claim 4 wherein the indicator is a visual indicator.

18. The method of claim 12 further comprising monitoring electrical brain activity of a plurality of persons participating in the medical procedure, wherein the electrical brain activity of each person is monitored via a electroencephalography monitoring device that provides electroencephalography data for each person; and
processing the electroencephalography data for each person to determine if the electroencephalography data for each person is outside an established range.

19. The method of claim 13 wherein the indicator is an indication of personnel dynamics between each of the plurality of persons.

20. A method of monitoring medical procedures, the method comprising:
identifying a plurality of steps in operating room flow that are critical to multiple operating room system management;
associating with each step in the plurality of steps a sensing mechanism that accurately tracks starting and ending times for each step;
reconstructing hand motions of a surgeon via a surgical tool global positioning system;
monitoring electrical brain activity of a plurality of persons participating in the medical procedure, wherein the electrical brain activity is monitored via a electroencephalography monitoring device that provides electroencephalography data;
processing electroencephalography data; and
processing the electroencephalography data for each person to determine if the electroencephalography data for each person is outside an established range.

21. The method of claim 20 further comprising reconstructing a network of the mental state of the plurality of persons participating in the medical procedure.
